SEQUENCE LISTING (54 sequences)

SEQ ID NO: 1 - DNA, 20 bp, Artificial Sequence
  Primer
agagggtgaaggtgatgcaa

SEQ ID NO: 2 - DNA, 20 bp, Artificial Sequence
  Primer
ccatgtgtaatcccagcagc

SEQ ID NO: 3 - DNA, 20 bp, Artificial Sequence
  Primer
taaacggccacaagttcagc

SEQ ID NO: 4 - DNA, 20 bp, Artificial Sequence
  Primer
cttgtacagctcgtccatgc

SEQ ID NO: 5 - DNA, 20 bp, Artificial Sequence
  Primer
agagggtgaaggtgatgcaa

SEQ ID NO: 6 - DNA, 20 bp, Artificial Sequence
  Primer
ctcttgaagaagtcgtgccg

SEQ ID NO: 7 - DNA, 20 bp, Artificial Sequence
  Primer
cacatgaagcagcacgactt

SEQ ID NO: 8 - DNA, 20 bp, Artificial Sequence
  Primer
tccttgaagtcgatgccctt

SEQ ID NO: 9 - DNA, 20 bp, Artificial Sequence
  Primer
agcaactgggatgacatgga

SEQ ID NO: 10 - DNA, 20 bp, Artificial Sequence
  Primer
catacatggcggggacattg

SEQ ID NO: 11 - DNA, 23 bp, Artificial Sequence
  TK ALS CRISPR Targets
tctaggtatggtcgttcaatggg

SEQ ID NO: 12 - DNA, 30 bp, Artificial Sequence
  TK ALS CRISPR Targets
gtcgttcaatgttttagagctagaaatagc

SEQ ID NO: 13 - DNA, 31 bp, Artificial Sequence
  TK ALS CRISPR Targets
cgttcaatgttttagagctagaaatagcaag

SEQ ID NO: 14 - DNA, 31 bp, Artificial Sequence
  TK ALS CRISPR Targets
catacctagaaatcgctatgtcgactctatc

SEQ ID NO: 15 - DNA, 33 bp, Artificial Sequence
  TK ALS CRISPR Targets
accatacctagaaatcgctatgtcgactctatc

SEQ ID NO: 16 - DNA, 23 bp, Artificial Sequence
  TK ALS CRISPR Targets
gtgttgcctatgatccccgccgg

SEQ ID NO: 17 - DNA, 29 bp, Artificial Sequence
  TK ALS CRISPR Targets
gatccccgcgttttagagctagaaatagc

SEQ ID NO: 18 - DNA, 33 bp, Artificial Sequence
  TK ALS CRISPR Targets
gtcgttcaatgttttagagctagaaatagcaag

SEQ ID NO: 19 - DNA, 32 bp, Artificial Sequence
  TK ALS CRISPR Targets
ataggcaacacaatcgctatgtcgactctatc

SEQ ID NO: 20 - DNA, 31 bp, Artificial Sequence; identical to SEQ ID NO: 14
  TK ALS CRISPR Targets

SEQ ID NO: 21 - DNA, 23 bp, Artificial Sequence
  TK ALS CRISPR Targets
ccatgaacccaccgccggcgggg

SEQ ID NO: 22 - DNA, 28 bp, Artificial Sequence
  TK ALS CRISPR Targets
cgccggcggttttagagctagaaatagc

SEQ ID NO: 23 - DNA, 33 bp, Artificial Sequence
  TK ALS CRISPR Targets
accgccggcggttttagagctagaaatagcaag

SEQ ID NO: 24 - DNA, 33 bp, Artificial Sequence
  TK ALS CRISPR Targets
gtgggttcatggaatcgctatgtcgactctatc

SEQ ID NO: 25 - DNA, 31 bp, Artificial Sequence
  TK ALS CRISPR Targets
gggttcatggaatcgctatgtcgactctatc

SEQ ID NO: 26 - DNA, 23 bp, Artificial Sequence
  TK ALS CRISPR Targets
ttgcctatgatccccgccggcgg

SEQ ID NO: 27 - DNA, 28 bp, Artificial Sequence
  TK ALS CRISPR Targets
cccgccgggttttagagctagaaatagc

SEQ ID NO: 28 - DNA, 33 bp, Artificial Sequence
  TK ALS CRISPR Targets
tccccgccgggttttagagctagaaatagcaag

SEQ ID NO: 29 - DNA, 33 bp, Artificial Sequence
  TK ALS CRISPR Targets
ttgcctatgatcaatcgctatgtcgactctatc

SEQ ID NO: 30 - DNA, 31 bp, Artificial Sequence
  TK ALS CRISPR Targets
tcataggcaaaatcgctatgtcgactctatc

SEQ ID NO: 31 - DNA, 23 bp, Artificial Sequence
  TK ALS CRISPR Targets
tccatgaacccaccgccggcggg

SEQ ID NO: 32 - DNA, 28 bp, Artificial Sequence
  TK ALS CRISPR Targets
ccgccggcgttttagagctagaaatagc

SEQ ID NO: 33 - DNA, 33 bp, Artificial Sequence
  TK ALS CRISPR Targets
caccgccggcgttttagagctagaaatagcaag

SEQ ID NO: 34 - DNA, 33 bp, Artificial Sequence
  TK ALS CRISPR Targets
tgggttcatggaaatcgctatgtcgactctatc

SEQ ID NO: 35 - DNA, 31 bp, Artificial Sequence
  TK ALS CRISPR Targets
ggttcatggaaatcgctatgtcgactctatc

SEQ ID NO: 36 - DNA, 23 bp, Artificial Sequence
  TK ALS CRISPR Targets
ccatcaccggccaagttccccgg

SEQ ID NO: 37 - DNA, 30 bp, Artificial Sequence
  TK ALS CRISPR Targets
ccaagttcccgttttagagctagaaatagc

SEQ ID NO: 38 - DNA, 31 bp, Artificial Sequence
  TK ALS CRISPR Targets
ccggtgatggaatcgctatgtcgactctatc

SEQ ID NO: 39 - DNA, 23 bp, Artificial Sequence
  TK ALS CRISPR Targets
cgatcattctccggggaacttgg

SEQ ID NO: 40 - DNA, 29 bp, Artificial Sequence
  TK ALS CRISPR Targets
cggggaactgttttagagctagaaatagc

SEQ ID NO: 41 - DNA, 32 bp, Artificial Sequence
  TK ALS CRISPR Targets
gagaatgatcgaatcgctatgtcgactctatc

SEQ ID NO: 42 - DNA, 23 bp, Artificial Sequence
  TK ALS CRISPR Targets
ctaccctggcggcgcatccatgg

SEQ ID NO: 43 - DNA, 29 bp, Artificial Sequence
  TK ALS CRISPR Targets
gcgcatccagttttagagctagaaatagc

SEQ ID NO: 44 - DNA, 31 bp, Artificial Sequence
  TK ALS CRISPR Targets
gccagggtagaatcgctatgtcgactctatc

SEQ ID NO: 45 - DNA, 23 bp, Artificial Sequence
  TK ALS CRISPR Targets
atctccatggatgcgccgccagg

SEQ ID NO: 46 - DNA, 28 bp, Artificial Sequence
  TK ALS CRISPR Targets
gcgccgccgttttagagctagaaatagc

SEQ ID NO: 47 - DNA, 33 bp, Artificial Sequence
  TK ALS CRISPR Targets
atccatggagataatcgctatgtcgactctatc

SEQ ID NO: 48 - DNA, 23 bp, Artificial Sequence
  TK ALS CRISPR Targets
cctatgatccccgccggcggtgg

SEQ ID NO: 49 - DNA, 28 bp, Artificial Sequence
  TK ALS CRISPR Targets
gccggcgggttttagagctagaaatagc

SEQ ID NO: 50 - DNA, 33 bp, Artificial Sequence
  TK ALS CRISPR Targets
ggggatcataggaatcgctatgtcgactctatc

SEQ ID NO: 51 - DNA, 23 bp, Artificial Sequence
  TK ALS CRISPR Targets
ttgcctatgatccccgccggcgg

SEQ ID NO: 52 - DNA, 29 bp, Artificial Sequence
  TK ALS CRISPR Targets
ccccgccgggttttagagctagaaatagc

SEQ ID NO: 53 - DNA, 32 bp, Artificial Sequence
  TK ALS CRISPR Targets
atcataggcaaaatcgctatgtcgactctatc

SEQ ID NO: 54 - DNA, 8714 bp, Artificial Sequence
  Vector
tcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggcgccattcgccattcaggctgcgcaactgttgggaagggcgatcggtgcgggcctcttcgctattacgccagttgccatcattgagtttggaaccctgaacagactgccggtgataagccggaggaaggtgaggatgacgtcaagtcatcatgccccttatgccctgggcgacacacgtgctacaatggccgggacaaagggtcgcgatcccgcgagggtgagctaactccaaaaacccgtcctcagttcggattgcaggctgcaactcgcctgcatgaagccggaatcgctagtaatcgccggtcagccatacggcggtgaatccgttcccgggccttgtacacaccgcccgtcacactatgggagctggccatgcccgaagtcgttaccttaaccgcaaggagggggatgccgaaggcagggctagtgactggagtgaagtcgtaacaaggtagccgtactggaaggtgcggctggatcacctccttttcagggagagctaatgcttgttgggtattttggtttgacactgcttcacacccaaaaagaagggagctacgtctgagttaaacttggagatggaagtcttctttcgtttctcgacagtgaagtaagaccaagctcatgagcttattatctcaggtcggaacaagttgataggatccccctttttacgtccccatgccccctgtgtggcgacatgggggcgaaaaaaggaaagagagggatggggtttctctcgcttttggcatagtgggcccccagtggggggctcgcacgacgggctattagctcagtgggtagagcgcgcccctgataattgcgtcgttgtgcctgggctgtgagggctctcagccacatggatagttcaatgtgctcatcggcgcctgaccctgagatgtggatcatccaaggcacattagcatggcgtactcctcctgttcgaaccggggtttgaaaccaaacttctcctcaggaggatagatggggcgattcaggtgagatccaatgtagatccaactttcgattcactcgtgggatccgggcggtccgggggggaccaccatggctcctctcttctcgagaatccatacatcccttatcagtgtatggacagctatctctcgagcacaggtttaggttcggcctcaatgggaaaataaaatggagcacctaacaacgcatcttcacagaccaagaactacgagatcacccctttcattctggggtgacggagggatcataccattcgagccttttttttttcatgcttttccccgaggtctggagaaagctgaaatcaatgggatgtgtctatttatctatctcttgactcgaaatgggagcaggtttgaaaaaggatcttagagtgtctagggttgggccaggagggtctcttaacgccttcttttttcttctcatcggattcacaaagacttgccatggtaaggaagaaggggagaacaggcacacttggagagcgcagtacaacggagagttgtatgctgcgttcgggaaggatgaatcgctcccgaaaaggaatctattgattctctcccaattggttggaccgtaggtgcgatgatttacttcacgggcgaggtctctggttcaagtccaggatggcccaggaagttatgggccgcaatgtgagtttttgtagttggatttgctcccccgccgtcgttcaatgagaatggataagaggctcgtgggattgacgtgagggggcagggatggctatatttctgggagcgaactccgggcgaatgagaccacaacggtttcccactagaaataattttgtttaactttaagaaggagatatacggcgcgccatggtagatctgactagtaaaggagaagaacttttcactggagttgtcccaattcttgttgaattagatggtgatgttaatgggcacaaattttctgtcagtggagagggtgaaggtgatgcaacatacggaaaacttacccttaaatttatttgcactactggaaaactacctgttccgtggccaacacttgtcactactttctcttatggtgttcaatgcttttcaagatacccagatcatatgaagcggcacgacttcttcaagagcgccatgcctgagggatacgtgcaggagaggaccatcttcttcaaggacgacgggaactacaagacacgtgctgaagtcaagtttgagggagacaccctcgtcaacaggatcgagcttaagggaatcgatttcaaggaggacggaaacatcctcggccacaagttggaatacaactacaactcccacaacgtatacatcatggccgacaagcaaaagaacggcatcaaagccaacttcaagacccgccacaacatcgaagacggcggcgtgcaactcgctgatcattatcaacaaaatactccaattggcgatggccctgtccttttaccagacaaccattacctgtccacacaatctgccctttcgaaagatcccaacgaaaagagagaccacatggtccttcttgagtttgtaacagctgctgggattacacatggcatggatgaactatacaaagctagccaccaccaccaccaccacgtgtgagcgatcgcagttgtagggagggatccttaattaaatgagcccagaacgacgcccggccgacatccgccgtgccaccgaggcggacatgccggcggtctgcaccatcgtcaaccactacatcgagacaagcacggtcaacttccgtaccgagccgcaggaaccgcaggagtggacggacgacctcgtccgtctgcgggagcgctatccctggctcgtcgccgaggtggacggcgaggtcgccggcatcgcctacgcgggcccctggaaggcacgcaacgcctacgactggacggccgagtcgaccgtgtacgtctccccccgccaccagcggacgggactgggctccacgctctacacccacctgctgaagtccctggaggcacagggcttcaagagcgtggtcgctgtcatcgggctgcccaacgacccgagcgtgcgcatgcacgaggcgctcggatatgccccccgcggcatgctgcgggcggccggcttcaagcacgggaactggcatgacgtgggtttctggcagctggacttcagcctgccggtaccgccccgtccggtcctgcccgtcaccgagatttgaaagcttgaaattcaattaaggaaataaattaaggaaatacaaaaaggggggtagtcatttgtatataactttgtatgacttttctcttctatttttttgtatttcctccctttccttttctatttgtatttttttatcattgcttccattgaattccgtgttctgtgaataacttcgtatagcatacattatacgaagttatgagaagtccgtatttttccaatcaacttcattaaaaatttgaatagatctacatacaccttggttgacacgagtatataagtcatgttatactgttgaataacaagccttccattttctattttgatttgtagaaaactagtgtgcttgggagtccctgatgattaaataaaccaagatttttctagacatatgggtcgacatggaacagaagttgatttccgaagaagaccccgagtagtcgggaggatggcagaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataatgactcgaggcggccgcctgcaggtgctattgctcctttctttttttctttttatttatttactggtattttacttacatagacttttttgtttacattatagaaaaagaaggagaggttattttcttgcatttattcatgattgagtattctattttgattttgtatttgtttgggctgcgggtcaactgcccctatcggaaataggattgactaccgattccgaaggaactggagttacatctcttttccattcaagagttcttatgcgtttccacgcccctttgagaccccgaaaaatggacaaattccttttcttaggaacacatacaagattcgtcactacaaaaaggataatggtaacctgcgccagggaaaagaatgggcccggggatatagctcagctggtagagcgctgcccttgcaaggcagatgtcagcggttcgagtccgcttatctccaccactgcgccagggaaaagaatagaagaagcgtctgactccttcatgcatgctccacttggctcggggggatatagctcagttggtagagctccgctcttgcaattgggtcgttgcgattacgggttggatgtctaattgtccaggcggtaatgatagtatcttgtacctgaaccggtggctcactttttctaagtaatggggaagaggaccgaaacatgccactgaaagactctactgagacaaagatgggctgtcaagaacgtcaagaacgtagaggaggtaggatgggcagttggtcagatctagtatggatcgtacatggacggtagttggagtcggcggctctcctagggttcccttatcggggatccctggggaagaggatcaagttggcccttgcgaacagcttgatgcactatctcccttcaaccctttgagcgaaatgcggcaaaaggaaggaaaatccatggaccgaccccatcatctccaccccgtaggaactacgagattaccccaaggacgccttcggcatccaggggtcacggaccgaccatagaaccctgttcaataagtggaacgcattagctgtccgctctcaggttgggcagtaagggtcggagaagggcaatcactcattcttaaaaccagcgttcttaaggccaaagagtcggcggaaaaggggggaaagctctccgttcctggtttcctgtagctggatcctccggaaccacaagaatccttagttagaatgggattccaactcagcaccttttgagtgagattttgagaagagttgctctttggagagcacagtacgatgaaagttgtaagctgtgttcgggggggagttattgtctatcgttggcctctatggtagaatcagtcgggggacctgagaggcggtggtttaccctgcggcggatgtcagcggttcgagtccgcttatctccaactcgtgaacttagccgatacaaagctatatgatagcacccaatttttccgattcggcggttcgatctatgatttatcattcatggacgttgataagatccatccatttagcagcaccttaggatggcatagccttaaaattaagggcgaggttcaaacgaggaaaggcttacggtggatacctaggcacccagagacgaggaagggcgtagtaagcgacgaaatgcttcggggagttgaaaataagcatagatccggagattcccgaataggttaacctttcaaactgctgctgaatccatgggcaggcaagagacaacctggcgaactgaaacatcttagtagccagaggaaaagaaagcaaaagcgattcccgtagtagcggcgagcgaaatgggagcagcctaaaccgtgaaaacggggttgtgggagagcaatacaagcgtcgtgctgctaggcgaagcagtagaatgctgcaccctagatggcgaaagtccagtagccgaaagcatcactagcttacgctctgacccgagtagcatggggcacgtggaatcccgtgtgaatcagcaaggaccaccttgctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtc

(19) United States
(12) Patent Application Publication
(10) Pub. No.: US 2017/0314033 A1
(43) Pub. Date: Nov. 2, 2017

(54) HERBICIDE-RESISTANT TARAXACUM KOK-SAGHYZ AND TARAXACUM BREVICORNICULATUM

(71) Applicant: Ohio State Innovation Foundation, Columbus, OH (US)

(72) Inventors: Katrina Cornish, Wooster, OH (US); Kyle Arthur Benzle, Lakeville, OH (US); Lu Zhao, Wooster, OH (US); Yingxiao Zhang, Columbus, OH (US); Brian Iaffaldano, Columbus, OH (US)

(21) Appl. No.: 15/496,946

(22) Filed: Apr. 25, 2017

Related U.S. Application Data
(60) Provisional application No. 62/330,675, filed on May 2, 2016.

(51) Int. Cl. C12N 15/82; C12N 9/10; A01H 5/00; C12N 9/00

(52) U.S. Cl. CPC C12N 15/8241; C12N 15/8205; C12N 9/10; A01H 5/00; C12N 9/00; C12N 15/82

(57) ABSTRACT

The invention provides the genetically manipulated herbicide-resistant rubber producing dandelion plants and seed of said plants. Another aspect of the invention comprises progeny plants, or seeds, or regenerable parts of plants and seeds of the genetically manipulated herbicide-resistant dandelion plants. Applicants have further found that use of root cells for transformation and other optimized protocols enable quick transformation with high plant regeneration. Further, Applicants have developed the first transformation/regeneration protocol that is successful without the addition of hormone treatment.

### CROSS REFERENCE TO RELATED APPLICATION

This application claims priority under 35 U.S.C. §119 to provisional application Ser. No. 62/330,675, filed May 2, 2016, herein incorporated by reference in its entirety.

### FIELD OF THE INVENTION

The invention relates to herbicide resistant dandelions. Specifically, herbicide resistance in rubber-producing dandelion species (e.g., Taraxacum kok-saghyz and Taraxacum brevicorniculatum). Disclosed herein are methods of producing genetically manipulated plants with increased herbicide resistance, particularly modified target gene sequences, and/or integration of exogenous sequences, which prevent herbicide susceptibility but retain normal plant development, polynucleotides for engineering the same, and genetically manipulated plants and seeds generated therefrom.

### BACKGROUND OF THE INVENTION

Natural rubber (NR, cis-1,4-polyisoprene) is a critical strategic resource for manufacturing at least 40,000 products, including tires, gloves, condoms and medical devices. Worldwide natural rubber production is almost exclusively reliant on the Brazilian or Para rubber tree (Hevea brasiliensis Muell. Arg.), which is cultivated mostly in the equatorial regions of Southeast Asia. Unfortunately, H. brasiliensis cultivation is threatened by South American leaf blight (SALB), a fungal disease caused by Microcyclus ulei, which inhibits NR production on a commercial scale in South and Central America. Moreover, Hevea rubber production must also contend with rising costs of labor, land competition with palm plantations, and increased evidence of life threatening allergenic reactions to NR latex. Accordingly, there is an imperative need to develop alternative rubber resources and expedite their commercialization to meet market demands.

Research and development programs for rubber production have identified around 2500 plant species that are able to produce NR, although very few can produce commercially-viable amounts of high quality rubber. Taraxacum kok-saghyz (TK; Kazak dandelion) and its vigorous apomictic cousin, Taraxacum brevicorniculatum (TB) are rubber-producing dandelion species under development as potential crops and model systems of rubber biosynthesis. The former is of industrial interest, as it produces a high percentage of high quality rubber in its roots; the latter is of interest as a model system for rubber biosynthesis and a source of vigor in breeding efforts. TK was discovered in Kazakhstan in 1931 and was cultivated over 1000 acres in the United States throughout World War II (WWII) to alleviate NR shortages, nonetheless it could not compete economically with Heave rubber as availability was restored after WWII. The main reasons for non-economic production of rubber in TK was its poor agronomic performance, as 50-70% of the production costs in the WWII emergency project were due to tilling of weeds. Despite the ongoing research in this field, barriers impeding the large scale commercialization of rubber-producing dandelions in the conventional farm and crop rotation systems remain, namely significantly reduced crop yields as a result of uncontrolled weeds.

Thus, a long standing need exists in the art for rubber-producing dandelions that are resistant to herbicides.

### SUMMARY OF THE INVENTION

Applicants have produced methods and compositions related to genetically manipulated herbicide-resistant dandelion plants, and progeny and populations thereof are provided herein.

In one aspect, this invention provides the genetically manipulated herbicide-resistant rubber producing dandelion plants and seed of said plants. Another aspect of the invention comprises progeny plants, or seeds, or regenerable parts of plants and seeds of the genetically manipulated herbicide-resistant dandelion plants. According to the invention, Applicants have surprisingly discovered that the rubber producing species of dandelion (Taraxacum kok-saghyz and Taraxacum brevicorniculatum) are incompatible and do not cross with the traditional dandelion weed, (Taraxacum officinale) making the generation of herbicide resistant plants for mass production possible without any deleterious cross breeding effects for those who still wish to eradicate the common weed species. Applicants have further found that use of root cells for transformation and other optimized protocols enable quick transformation with high plant regeneration and they have developed the first transformation/regeneration protocol that is successful without the addition of hormone treatment.

The present invention is also directed to a nucleus of a dandelion cell, wherein said nucleus comprises a chromosome having a heterologous polynucleotide insert that provides for improved herbicide tolerance, for example, herbicide resistance genes, including, but not limited to glyphosphate-, ALS- (imidazoline, sulfonylurea), aryloxyalkanoate-, and HPPD-, PPO-, and glufosinate-resistance genes. Of particular interest is a chromosome wherein the heterologous polynucleotide comprises a promoter for expression of a polynucleotide conferring herbicide resistance, and wherein said promoter is adjacent to dandelion genomic sequence. In certain embodiments, a dandelion chromosome comprising a heterologous transgenic insert comprising a promoter that is operably linked to a polynucleotide conferring herbicide resistance is provided. A 5′ terminus of the heterologous transgenic insert can overlap a 3′ terminus of dandelion genomic sequence in certain embodiments.

In one exemplary embodiment, the polynucleotide conferring herbicide tolerance is the bar gene, ensuring dandelions that are tolerant to glufosinate. Therefore, weeds in the fields where such dandelion plants are grown can be controlled by application of herbicides comprising glufosinate as an active ingredient (such as Liberty′.). In certain embodiments, a chromosome of the invention is located within a dandelion cell that also contains a second unlinked heterologous polynucleotide. Plants or seed comprising any of the dandelion chromosomes of the invention are also provided.

Another aspect of the invention provides herbicide-resistant plants through gene targeting, and targeted genomic modification in dandelions. In particular, the methods and compositions of the invention allow for exogenous transgenic insertion and/or genomic modification of an endogenous gene, in which the genomic modification produces a mutation in the endogenous gene such that the endogenous gene produces a product that results in an herbicide tolerant plant.

In a preferred embodiment, genetically modified herbicide-resistant dandelions exploit known mutations in an endogenous gene such as known mutations in the ALS gene (acetolactate synthase (ALS), also known as acetohydroxyacid synthase (AHAS)) that confer tolerance to Group B herbicides, or ALS inhibitor herbicides such as imidazolinone or sulfonylurea.

According to one aspect of the invention, exogenous sequence and/or to stack traits that exploit differential selection at an endogenous locus (e.g., ALS locus) in dandelion genomes. The strategy facilitates generation of plants that have one or more transgenes (or one or more genes of interest (GOI), positioned precisely at an endogenous plant locus. The methods and compositions described herein enable both parallel and sequential transgene stacking in dandelion genomes at precisely the same genomic location, including simultaneous editing of multiple alleles across multiple genomes of polyploid dandelion species. Also provided are cells (e.g., seeds), cell lines, organisms (e.g., plants), etc. comprising these transgene-stacked and/or simultaneously-modified alleles.

Another embodiment of the invention provides transgenic and/or targeted genomic editing (insertions, deletions, mutations, transgene stacking) which result, for example, in increased crop yield, a protein encoding disease resistance, a protein that increases growth, a protein encoding insect resistance, a protein encoding herbicide tolerance and the like. Increased yield can include, for example, increased biomass of the plant, larger plants, increased dry weight, increased solids context, higher total weight at harvest, enhanced intensity and/or uniformity of color of the crop, altered chemical (e.g., oil, fatty acid, carbohydrate, protein) characteristics, etc.

Thus, in one aspect, disclosed herein are methods for genomic modification (e.g., transgene stacking) at one or more endogenous alleles of a dandelion gene. The methods use root cells as the transformation target to drastically increase the number of transformants and have also developed techniques that allow for successful transformation and regeneration of plants without the need for hormone treatment. In certain embodiments, the transgene(s) is (are) integrated into an endogenous locus of a dandelion genome (e.g., polyploid plant). Transgene integration includes integration of multiple transgenes, which may be in parallel (simultaneous integration of one or more transgenes into one or more alleles) or sequential. In certain embodiments, the transgene does not include a transgenic marker, but is integrated into an endogenous locus that is modified upon integration of the transgene comprising a trait, for example, integration of the transgene(s) into an endogenous ALS locus such that the transgene is expressed and the ALS locus is modified to alter herbicide tolerance (e.g., Group B herbicides, or ALS inhibitor herbicides such as imidazolinone or sulfonylurea).

The transgene(s) is (are) integrated in a targeted manner using one or more non-naturally occurring nucleases, for example zinc finger nucleases, meganucleases, TALENs and/or a CRISPR/Cas system with an engineered single guide RNA. The transgene can comprise one or more coding sequences (e.g., proteins), non-coding sequences and/or may produce one or more RNA molecules (e.g., mRNA, RNAi, siRNA, shRNA, etc.). In certain embodiments, the transgene integration is simultaneous (parallel). Furthermore, any of the plant cells described herein may further comprise one or more additional transgenes, in which the additional transgenes are integrated into the genome at a different locus (or different loci) than the target allele(s) for transgene stacking. Thus, a plurality of endogenous loci may include integrated transgenes in the cells described herein.

Another aspect of the invention, disclosed herein are methods of breeding herbicide-resistant dandelions of the invention comprising crossing a dandelion plant of the invention with a second dandelion plant to yield a herbicide tolerant dandelion progeny, wherein at least partial herbicide resistance is introgressed from the dandelion plant of the invention into the second dandelion plant.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### DESCRIPTION OF THE FIGURES

FIGS. 1A-1B show binary vectors for green fluorescent protein (GFP) and cyan fluorescent protein (CFP) expression. FIG. 1A shows the structure of pEG-35S::GFP construct. FIG. 1B shows the structure of pEG-35S::CFP construct. Kanamycin resistance gene nptII was controlled by Ti plasmid mannopine synthase (MAS) promoter and terminator. GFP and CFP were regulated by CaMV 35 S promoter and octopine synthase (OCS) terminator. Black arrows (→) indicate the transcription direction of each gene. PCR amplified regions are shown as gray arrows (→).

FIG. 2A-2D shows the effects of different explants (leaf disc and root) and three media (½ MS, MS+BAP and MS+BAP+IAA) on Taraxacum kok-saghyz (TK) and T brevicorniculatum (TB) regeneration efficiency. FIG. 2A shows the regeneration efficiency of TK from leaf discs. Inserted photograph shows the regenerated shoots. FIG. 2B shows the regeneration efficiency of TK from root fragments. Inserted photograph shows the regenerated shoots using ½ MS medium. FIG. 2C shows the regeneration efficiency of TB from leaf discs. Inserted photograph shows the regenerated shoots. FIG. 2D shows the regeneration efficiency of TB from root fragments. Inserted photograph shows the regenerated shoots using ½ MS medium. Regeneration efficiency was calculated by dividing the number of regenerated calli or shoots by the number of starting leaf discs or root fragments. Callus regeneration efficiency is indicated by the light gray bar and shoots regeneration efficiency is indicated by the dark gray bar. Vertical bars indicate standard errors (SE). Statistical analysis was carried out using one-way ANOVA with the medium as the treatment. Comparison was conducted with same explants and within species. Mean±SE followed by same lower or uppercase letters are not significantly different for their respective data set according to Tukey's HSD at P<0.05.

FIG. 3A-3B shows the effects of inoculation and root size on Taraxacum kok-saghyz (TK) and T. brevicorniculatum (TB) regeneration efficiency. FIG. 2A shows the plant regeneration efficiency of TK and TB from root fragments without and with inoculation. Regeneration efficiency without inoculation is indicated by the light gray bar ( ) and regeneration efficiency with inoculation is indicated by the dark gray bar. FIG. 2B shows the plant regeneration efficiency of TK and TB from root fragments with diameter D≧1 mm and D<1 mm. Regeneration efficiency from root D≧1 mm is indicated by the light gray bar and regeneration efficiency from root D<1 mm is indicated by the dark gray bar. Plant regeneration efficiency was calculated by dividing the number of regenerated plants by the number of starting root fragments. Vertical bars indicate standard errors. Stars indicate the significant differences between treatments within species according to Tukey's HSD at P<0.05.

FIGS. 4A-4L show the A. rhizogenes-mediated transformation of Taraxacum kok-saghyz (TK) and T. brevicorniculatum (TB) using root fragments as explants. FIG. 4A shows TK root fragments explants. FIG. 4B shows complete TK putative transgenic plants, including leaves and hairy roots, were regenerated on ½ MS medium without hormone addition under kanamycin selection. FIG. 4C shows a transgenic TK plant after 2 months of selection with hairy root phenotypes. FIG. 4D shows a 2-month-old non-transgenic TK plant. FIG. 4E shows transgenic TK plants regenerated from transgenic hairy roots. FIG. 4F shows a transgenic TK plant established in soil with hairy root phenotypes and flowers. FIG. 4G shows TB root fragments explants. FIG. 4H shows complete TB putative transgenic plants including leaves and hairy roots were regenerated. FIG. 4I shows a transgenic TB plant after 2 months of selection with hairy root phenotypes. FIG. 4J shows a 2-month-old non-transgenic TB plant. FIG. 4K Shows a transgenic TB plants regenerated from transgenic hairy roots. FIG. 4L shows a transgenic TB plant established in soil with hairy root phenotypes. Size bars represent 2 cm.

FIG. 5A-5B show Polymerase chain reaction (PCR) analysis of green fluorescent protein (GFP) and cyan fluorescent protein (CFP) in transgenic Taraxacum kok-saghyz (TK) and T. brevicorniculatum (TB) plants. FIG. 5A is PCR analysis of GFP in four independent transformants of each species. FIG. 5B is PCR analysis of CFP in four independent transformants of each species. Leaf tissue was used for PCR analysis. L, 100 bp DNA ladder from New England Biolabs Inc. P, positive plasmid control. W, negative wild type non-transgenic plants control. Each number indicates an independent transgenic event.

FIG. 6A-6B show reverse transcription polymerase chain reaction (RT-PCR) analysis of green fluorescent protein (GFP) and cyan fluorescent protein (CFP) expression. FIG. 6A is RT-PCR analysis of GFP in two independent transformants of each species. FIG. 6B is RT-PCR analysis of CFP in two independent transformants of each species. Leaf tissue was used for RT-PCR analysis. P, positive plasmid control. W, negative wild type non-transgenic plant control. Each number stands for an independent transgenic event. Endogenous gene β-actin (ACTB) was used as endogenous gene control for each RT-PCR reaction. β-actin (ACTB) was used as endogenous gene control for each RT-PCR reaction.

FIG. 7A-7P show stable green fluorescent protein (GFP) and cyan fluorescent protein (CFP) expression in transgenic Taraxacum kok-saghyz (TK) and T. brevicorniculatum (TB) under a Leica TCS SP5 Confocal Microscope. FIG. 7A-7D show GFP expression in root tissue (7A and 7B) and leaf tissue (7C and 7D) of non-transgenic (WT) and transgenic (GFP) TK. (7E-7H), GFP expression in root tissue (7E and 7F) and leaf tissue (7G and 7H) of non-transgenic (WT) and transgenic (GFP) TB. FIGS. 7I-7L show CFP expression in root tissue (7I and 7J) and leaf tissue (7K and 7L) of non-transgenic (WT) and transgenic (CFP) TK. FIGS. 7M-7P show CFP expression in root tissue (M and N) and leaf tissue (O and P) of non-transgenic (WT) and transgenic (CFP) TB. Size bars represent 50 μm. Leaf and root tissue used for microscopy was obtained from plants after 8 weeks of selection. The florescence intensity shown in figures is not quantitative.

FIG. 8A-8E show stable inheritance and segregation of hairy root phenotypes and fluorescent protein gene in Taraxacum kok-saghyz (TK) T₁ generation. FIG. 8A shows TK T₁ generation plant 6 weeks after germination with hairy root phenotypes. FIG. 8B shows TK T₁ generation plant 6 weeks after germination without hairy root phenotypes. FIG. 8C shows 3-month old TK T₁ generation plant grown under tissue culture conditions with hairy root phenotypes. FIG. 8D shows 3-month old TK T₁ generation plant grown under tissue culture conditions without hairy root phenotypes. FIG. 8E is polymerase chain reaction (PCR) analysis of cyan fluorescent protein (CFP) of TK T₁ generation plant. L, 100 bp DNA ladder from New England Biolabs Inc., W, negative wild type non-transgenic plants control. P, positive plasmid control. T₁-1,2,3, TK T₁ generation plants. Size bars represent 2 cm.

FIG. 9 shows an exemplary herbicide-resistant rubber-producing dandelion plant according to the present invention.

FIG. 10 shows an exemplary herbicide-resistant rubber-producing dandelion plant according to the present invention.

FIG. 11 shows a comparison of wild-type dandelions and exemplary herbicide-resistant rubber-producing dandelions after exposure to herbicide.

FIG. 12 shows plants repairing the double strand break by Non-Homologous End Joining (NHEJ) pathway. Nucleotide non-anonymous mutations contributing herbicide resistance could be created and selected.

FIG. 13 shows plants repairing the double strand break by Homology Directed Repair (HDR) pathway. A DNA repair template containing herbicide resistance mutations can be introduced.

FIG. 14 shows the schematic employed to generate dandelions with resistance to ALS inhibitors.

FIG. 15 is a plasmid map used to create the TKS plants and varieties of the invention.

### DETAILED DESCRIPTION OF THE INVENTION

The present invention now will be described more fully with reference to the accompanying examples. The invention may be embodied in many different forms and should not be construed as limited to the embodiments set forth in this application; rather, these embodiments are provided so that this disclosure will satisfy applicable legal requirements.

Many modifications and other embodiments of the invention will come to mind to one skilled in the art to which this invention pertains, having the benefit of the teachings presented in the descriptions and the drawings herein. As a result, it is to be understood that the invention is not to be limited to the specific embodiments disclosed and that modifications and other embodiments are intended to be included within the scope of the appended claims. Although specific terms are used in the specification, they are used in a generic and descriptive sense only and not for purposes of limitation.

### General

In order to provide a clear and consistent understanding of the specification and the claims, including the scope given to such terms, the following definitions are provided. Units, prefixes, and symbols may be denoted in their SI accepted form. Unless otherwise indicated, nucleic acids are written left to right in 5′ to 3′ orientation; amino acid sequences are written left to right in amino to carboxy orientation, respectively. Numeric ranges are inclusive of the numbers defining the range and include each integer within the defined range. Amino acids may be referred to herein by either their commonly known three letter symbols or by the one-letter symbols recommended by the IUPAC-IUB Biochemical nomenclature Commission. Nucleotides, likewise, may be referred to by their commonly accepted single-letter codes. Unless otherwise provided for, software, electrical, and electronics terms as used herein are as defined in The New IEEE Standard Dictionary of Electrical and Electronics Terms (5th edition, 1993). The terms defined below are more fully defined by reference to the specification as a whole.

Practice of the methods, as well as preparation and use of the compositions disclosed herein employ, unless otherwise indicated, conventional techniques in molecular biology, biochemistry, chromatin structure and analysis, computational chemistry, cell culture, recombinant DNA and related fields as are within the skill of the art. These techniques are fully explained in the literature. See, e.g., Sambrook et al. MOLECULAR CLONING: A LABORATORY MANUAL, 2d ed., Cold Spring Harbor Laboratory Press, 1989; 3d ed., 2001; Ausubel et al., CURRENT PROTOCOLS IN MOLECULAR BIOLOGY, John Wiley & Sons, New York, 1987 and periodic updates; the series METHODS IN ENZYMOLOGY, Academic Press, San Diego; Wolfe, CHROMATIN STRUCTURE AND FUNCTION, Third edition, Academic Press, San Diego, 1998; METHODS IN ENZYMOLOGY, Vol. 304, “Chromatin” (P. M. Wassarman and A. P. Wolffe, eds.), Academic Press, San Diego, 1999; and METHODS IN MOLECULAR BIOLOGY, Vol. 119, “Chromatin Protocols” (P. B. Becker, ed.) Humana Press, Totowa, 1999.

Unless otherwise specified, “a,” “an,” “the,” and “at least one” are used interchangeably and mean one or more than one.

The terms “nucleic acid,” “polynucleotide,” and “oligonucleotide” are used interchangeably and refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity; i.e., an analogue of A will base-pair with T.

The terms “polypeptide,” “peptide” and “protein” are used interchangeably to refer to a polymer of amino acid residues. The term also applies to amino acid polymers in which one or more amino acids are chemical analogues or modified derivatives of a corresponding naturally-occurring amino acids.

The term “introduced” in the context of inserting a nucleic acid into a cell, means “transfection” or “transformation” or “transduction” and includes reference to the incorporation of a nucleic acid into a eukaryotic or prokaryotic cell where the nucleic acid may be incorporated into the genome of the cell (e.g., chromosome, plasmid, plastid or mitochondrial DNA), converted into an autonomous replicon, or transiently expressed (e.g., transfected mRNA).

The term “conservatively modified variants” applies to both amino acid and nucleic acid sequences. With respect to particular nucleic acid sequences, “conservatively modified variants” refers to those nucleic acids which encode identical or conservatively modified variants of the amino acid sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations” and represent one species of conservatively modified variation. Every nucleic acid sequence herein that encodes a polypeptide also, by reference to the genetic code, describes every possible silent variation of the nucleic acid.

One of ordinary skill will recognize that each codon in a nucleic acid (except AUG, which is ordinarily the only codon for methionine; and UGG, which is ordinarily the only codon for tryptophan) can be modified to yield a functionally identical molecule. Accordingly, each silent variation of a nucleic acid which encodes a polypeptide of the present invention is implicit in each described polypeptide sequence and is within the scope of the present invention.

As used herein “promoter” includes reference to a region of DNA upstream from the start of transcription and involved in recognition and binding of RNA polymerase and other proteins to initiate transcription. A “plant promoter” is a promoter capable of initiating transcription in plant cells whether or not its origin is a plant cell. Exemplary plant promoters include, but are not limited to, those that are obtained from plants, plant viruses, and bacteria which comprise genes expressed in plant cells such as Agrobacterium or Rhizobium. Examples of promoters under developmental control include promoters that preferentially initiate transcription in certain tissues, such as leaves, roots, or seeds. Such promoters are referred to as “tissue preferred”. Promoters which initiate transcription only in certain tissue are referred to as “tissue specific”. A “cell type” specific promoter primarily drives expression in certain cell types in one or more organs, for example, vascular cells in roots or leaves. An “inducible” or “repressible” promoter is a promoter which is under environmental control. Examples of environmental conditions that may affect transcription by inducible promoters include anaerobic conditions or the presence of light. Tissue specific, tissue preferred, cell type specific, and inducible promoters constitute the class of “non-constitutive” promoters. A “constitutive” promoter is a promoter which is active under most environmental conditions.

“Binding” refers to a sequence-specific, non-covalent interaction between macromolecules (e.g., between a protein and a nucleic acid). Not all components of a binding interaction need be sequence-specific (e.g., contacts with phosphate residues in a DNA backbone), as long as the interaction as a whole is sequence-specific or conformation specific. Such interactions are generally characterized by a dissociation constant (K_{d}) of 10⁻⁶ M⁻¹ or lower. “Affinity” refers to the strength of binding: increased binding affinity being correlated with a lower K_{d}.

A “binding protein” is a protein that is able to bind non-covalently to another molecule. A binding protein can bind to, for example, a DNA molecule (a DNA-binding protein), an RNA molecule (an RNA-binding protein) and/or a protein molecule (a protein-binding protein). In the case of a protein-binding protein, it can bind to itself (to form homodimers, homotrimers, etc.) and/or it can bind to one or more molecules of a different protein or proteins. A binding protein can have more than one type of binding activity. For example, zinc finger proteins have DNA-binding, RNA-binding and protein-binding activity.

A “zinc finger DNA binding protein” (or binding domain) is a protein, or a domain within a larger protein, that binds DNA in a sequence-specific manner through one or more zinc fingers, which are regions of amino acid sequence within the binding domain whose structure is stabilized through coordination of a zinc ion. The term zinc finger DNA binding protein is often abbreviated as zinc finger protein or ZFP.

A “TALE DNA binding domain” or “TALE” is a polypeptide comprising one or more TALE repeat domains/units. The repeat domains are involved in binding of the TALE to its cognate target DNA sequence. A single “repeat unit” (also referred to as a “repeat”) is typically 33-35 amino acids and includes hypervariable diresidues at positions 12 and/or 13 referred to as the Repeat Variable Diresidue (RVD) involved in DNA-binding specificity. TALE repeats exhibit at least some sequence homology with other TALE repeat sequences within a naturally occurring TALE protein. See, e.g., U.S. Pat. No. 8,586,526.

Zinc finger binding and TALE domains can be “engineered” to bind to a predetermined nucleotide sequence. Non-limiting examples of methods for engineering zinc finger proteins are design and selection. A designed zinc finger protein is a protein not occurring in nature whose design/composition results principally from rational criteria. Rational criteria for design include application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496.

A “selected” zinc finger protein or TALE is a protein not found in nature whose production results primarily from an empirical process such as phage display, interaction trap or hybrid selection. See e.g., U.S. Pat. No. 8,586,526, U.S. Pat. No. 5,789,538; U.S. Pat. No. 5,925,523; U.S. Pat. No. 6,007,988; U.S. Pat. No. 6,013,453; U.S. Pat. No. 6,200,759; WO 95/19431; WO 96/06166; WO 98/53057; WO 98/54311; WO 00/27878; WO 01/60970 WO 01/88197 and WO 02/099084.

As used herein, “vector” includes reference to a nucleic acid used in transfection of a host cell and into which can be inserted a polynucleotide. Vectors are often replicons. Expression vectors permit transcription of a nucleic acid inserted therein.

The term “sequence” refers to a nucleotide sequence of any length, which can be DNA or RNA; can be linear, circular or branched and can be either single-stranded or double stranded. The term “donor sequence” refers to a nucleotide sequence that is inserted into a genome. A donor sequence can be of any length, for example between 2 and 10,000 nucleotides in length (or any integer value there between or thereabove), preferably between about 100 and 1,000 nucleotides in length (or any integer there between), more preferably between about 200 and 500 nucleotides in length.

A “homologous, non-identical sequence” refers to a first sequence which shares a degree of sequence identity with a second sequence, but whose sequence is not identical to that of the second sequence. For example, a polynucleotide comprising the wild-type sequence of a mutant gene is homologous and non-identical to the sequence to the sequence of the mutant gene. In certain embodiments, the degree of homology between the two sequences is sufficient to allow homologous recombination therebetween, utilizing normal cellular mechanisms. Two homologous non-identical sequences can be any length and their degree of non-homology can be as small as a single nucleotide (e.g., for correction of genomic point mutation by targeted homologous recombination) or as large as 10 or more kilobases (e.g., for insertion of a gene at a predetermined ectopic site in a chromosome). Two polynucleotides comprising the homologous non-identical sequences need not be the same length. For example, an exogenous polynucleotide (i.e., donor polynucleotide) of between 20 and 10,000 nucleotides or nucleotide pairs can be used.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively.

Two or more sequences (polynucleotide or amino acid) can be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present disclosure is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects sequence identity. Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST. GenBank® is the recognized United States-NIH genetic sequence database, comprising an annotated collection of publicly available DNA sequences, and which further incorporates submissions from the European Molecular Biology Laboratory (EMBL) and the DNA DataBank of Japan (DDBJ), see Nucleic Acids Research, January 2013, v 41(D1) D36-42 for discussion. With respect to sequences described herein, the range of desired degrees of sequence identity is approximately 80% to 100% and any integer value therebetween. Typically the percent identities between sequences are at least 70-75%, preferably 80-82%, more preferably 85-90%, even more preferably 92%, still more preferably 95%, and most preferably 98% sequence identity.

Alternatively, the degree of sequence similarity between polynucleotides can be determined by hybridization of polynucleotides under conditions that allow formation of stable duplexes between homologous regions, followed by digestion with single-stranded-specific nuclease(s), and size determination of the digested fragments. Two nucleic acid, or two polypeptide sequences are substantially homologous to each other when the sequences exhibit at least about 70%-75%, preferably 80%-82%, more preferably 85%-90%, even more preferably 92%, still more preferably 95%, and most preferably 98% sequence identity over a defined length of the molecules, as determined using the methods above. As used herein, substantially homologous also refers to sequences showing complete identity to a specified DNA or polypeptide sequence. DNA sequences that are substantially homologous can be identified in a Southern hybridization experiment under, for example, stringent conditions, as defined for that particular system. Defining appropriate hybridization conditions is within the skill of the art. See, e.g., Sambrook et al., supra; Nucleic Acid Hybridization: A Practical Approach, editors B. D. Hames and S. J. Higgins, (1985) Oxford; Washington, D.C.; IRL Press).

Selective hybridization of two nucleic acid fragments can be determined as follows. The degree of sequence identity between two nucleic acid molecules affects the efficiency and strength of hybridization events between such molecules. A partially identical nucleic acid sequence will at least partially inhibit the hybridization of a completely identical sequence to a target molecule. Inhibition of hybridization of the completely identical sequence can be assessed using hybridization assays that are well known in the art (e.g., Southern (DNA) blot, Northern (RNA) blot, solution hybridization, or the like, see Sambrook, et al., Molecular Cloning: A Laboratory Manual, Second Edition, (1989) Cold Spring Harbor, N.Y.). Such assays can be conducted using varying degrees of selectivity, for example, using conditions varying from low to high stringency. If conditions of low stringency are employed, the absence of non-specific binding can be assessed using a secondary probe that lacks even a partial degree of sequence identity (for example, a probe having less than about 30% sequence identity with the target molecule), such that, in the absence of non-specific binding events, the secondary probe will not hybridize to the target.

When utilizing a hybridization-based detection system, a nucleic acid probe is chosen that is complementary to a reference nucleic acid sequence, and then by selection of appropriate conditions the probe and the reference sequence selectively hybridize, or bind, to each other to form a duplex molecule. A nucleic acid molecule that is capable of hybridizing selectively to a reference sequence under moderately stringent hybridization conditions typically hybridizes under conditions that allow detection of a target nucleic acid sequence of at least about 10-14 nucleotides in length having at least approximately 70% sequence identity with the sequence of the selected nucleic acid probe. Stringent hybridization conditions typically allow detection of target nucleic acid sequences of at least about 10-14 nucleotides in length having a sequence identity of greater than about 90-95% with the sequence of the selected nucleic acid probe. Hybridization conditions useful for probe/reference sequence hybridization, where the probe and reference sequence have a specific degree of sequence identity, can be determined as is known in the art (see, for example, Nucleic Acid Hybridization: A Practical Approach, editors B. D. Hames and S. J. Higgins, (1985) Oxford; Washington, D.C.; IRL Press).

Conditions for hybridization are well-known to those of skill in the art. Hybridization stringency refers to the degree to which hybridization conditions disfavor the formation of hybrids containing mismatched nucleotides, with higher stringency correlated with a lower tolerance for mismatched hybrids. Factors that affect the stringency of hybridization are well-known to those of skill in the art and include, but are not limited to, temperature, pH, ionic strength, and concentration of organic solvents such as, for example, formamide and dimethylsulfoxide. As is known to those of skill in the art, hybridization stringency is increased by higher temperatures, lower ionic strength and lower solvent concentrations.

With respect to stringency conditions for hybridization, it is well known in the art that numerous equivalent conditions can be employed to establish a particular stringency by varying, for example, the following factors: the length and nature of the sequences, base composition of the various sequences, concentrations of salts and other hybridization solution components, the presence or absence of blocking agents in the hybridization solutions (e.g., dextran sulfate, and polyethylene glycol), hybridization reaction temperature and time parameters, as well as, varying wash conditions. The selection of a particular set of hybridization conditions is selected following standard methods in the art (see, for example, Sambrook, et al., Molecular Cloning: A Laboratory Manual, Second Edition, (1989) Cold Spring Harbor, N.Y.).

The term “substantial identity” of polynucleotide sequences means that a polynucleotide comprises a sequence that has at least 70% sequence identity, preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of at least 60%, or preferably at least 70%, 80%, 90%, and most preferably at least 95%.

Another indication that nucleotide sequences are substantially identical is if two molecules hybridize to each other under stringent conditions. However, nucleic acids which do not hybridize to each other under stringent conditions are still substantially identical if the polypeptides which they encode are substantially identical. This may occur, e.g., when a copy of a nucleic acid is created using the maximum codon degeneracy permitted by the genetic code. One indication that two nucleic acid sequences are substantially identical is that the polypeptide which the first nucleic acid encodes is immunologically cross reactive with the polypeptide encoded by the second nucleic acid.

“Recombination” refers to a process of exchange of genetic information between two polynucleotides. For the purposes of this disclosure, “homologous recombination (HR)” refers to the specialized form of such exchange that takes place, for example, during repair of double-strand breaks in cells. This process requires nucleotide sequence homology, that uses a “donor” molecule to template repair of a “target” molecule (i.e., the one that experienced the double-strand break), and is variously known as “non-crossover gene conversion” or “short gene conversion,” because it leads to the transfer of genetic information from the donor to the target. Without wishing to be bound by any particular theory, such transfer can involve mismatch correction of heteroduplex DNA that forms between the broken target and the donor, and/or “synthesis-dependent strand annealing,” in which the donor is used to resynthesize genetic information that will become part of the target, and/or related processes. Such specialized HR often results in an alteration of the sequence of the target molecule such that part or all of the sequence of the donor polynucleotide is incorporated into the target polynucleotide.

“Cleavage” refers to the breakage of the covalent backbone of a DNA molecule. Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events. DNA cleavage can result in the production of either blunt ends or staggered ends. In certain embodiments, fusion polypeptides are used for targeted double-stranded DNA cleavage.

A “cleavage domain” comprises one or more polypeptide sequences which possess catalytic activity for DNA cleavage. A cleavage domain can be contained in a single polypeptide chain or cleavage activity can result from the association of two (or more) polypeptides.

A “cleavage half-domain” is a polypeptide sequence which, in conjunction with a second polypeptide (either identical or different) forms a complex having cleavage activity (preferably double-strand cleavage activity). The terms “first and second cleavage half-domains;” “+ and − cleavage half-domains” and “right and left cleavage half-domains” are used interchangeably to refer to pairs of cleavage half-domains that dimerize.

An “engineered cleavage half-domain” is a cleavage half-domain that has been modified so as to form obligate heterodimers with another cleavage half-domain (e.g., another engineered cleavage half-domain). See, also, U.S. Patent Publication Nos. 2005/0064474, 20070218528, 2008/0131962 and 2011/0201055, incorporated herein by reference in their entireties.

A “chromosome,” is a chromatin complex comprising all or a portion of the genome of a cell. The genome of a cell is often characterized by its karyotype, which is the collection of all the chromosomes that comprise the genome of the cell. The genome of a cell can comprise one or more chromosomes. “Chromatin” is the nucleoprotein structure comprising the cellular genome. Cellular chromatin comprises nucleic acid, primarily DNA, and protein, including histones and non-histone chromosomal proteins. The majority of eukaryotic cellular chromatin exists in the form of nucleosomes, wherein a nucleosome core comprises approximately 150 base pairs of DNA associated with an octamer comprising two each of histones H2A, H2B, H3 and H4; and linker DNA (of variable length depending on the organism) extends between nucleosome cores. A molecule of H1 is generally associated with the linker DNA. For purposes of the present disclosure, the term “chromatin” is meant to encompass all types of cellular nucleoprotein, both prokaryotic and eukaryotic. Cellular chromatin includes both chromosomal and episomal chromatin.

An “accessible region” is a site in cellular chromatin in which a target site present in the nucleic acid can be bound by an exogenous molecule which recognizes the target site. Without wishing to be bound by any particular theory, it is believed that an accessible region is one that is not packaged into a nucleosomal structure. The distinct structure of an accessible region can often be detected by its sensitivity to chemical and enzymatic probes, for example, nucleases.

A “target site” or “target sequence” is a nucleic acid sequence that defines a portion of a nucleic acid to which a binding molecule will bind, provided sufficient conditions for binding exist. For example, the sequence 5′-GAATTC-3′ is a target site for the EcoRI restriction endonuclease.

An “exogenous” molecule is a molecule that is not normally present in a cell, but can be introduced into a cell by one or more genetic, biochemical or other methods. “Normal presence in the cell” is determined with respect to the particular developmental stage and environmental conditions of the cell. Thus, for example, a molecule that is present in cells only during the early stages of development of a flower is an exogenous molecule with respect to the cells of a fully developed flower. Similarly, a molecule induced by heat shock is an exogenous molecule with respect to a non-heat-shocked cell. An exogenous molecule can comprise, for example, a coding sequence for any polypeptide or fragment thereof, a functioning version of a malfunctioning endogenous molecule or a malfunctioning version of a normally-functioning endogenous molecule. Additionally, an exogenous molecule can comprise a coding sequence from another species that is an ortholog of an endogenous gene in the host cell.

An exogenous molecule can be, among other things, a small molecule, such as is generated by a combinatorial chemistry process, or a macromolecule such as a protein, nucleic acid, carbohydrate, lipid, glycoprotein, lipoprotein, polysaccharide, any modified derivative of the above molecules, or any complex comprising one or more of the above molecules. Nucleic acids include DNA and RNA, can be single- or double-stranded; can be linear, branched or circular; and can be of any length. Nucleic acids include those capable of forming duplexes, as well as triplex-forming nucleic acids. See, for example, U.S. Pat. Nos. 5,176,996 and 5,422,251. Proteins include, but are not limited to, DNA-binding proteins, transcription factors, chromatin remodeling factors, methylated DNA binding proteins, polymerases, methylases, demethylases, acetylases, deacetylases, kinases, phosphatases, integrases, recombinases, ligases, topoisomerases, gyrases and helicases. Thus, the term includes “transgenes” or “genes of interest” which are exogenous sequences introduced into a plant cell.

An exogenous molecule can be the same type of molecule as an endogenous molecule, e.g., an exogenous protein or nucleic acid. For example, an exogenous nucleic acid can comprise an infecting viral genome, a plasmid or episome introduced into a cell, or a chromosome that is not normally present in the cell. Methods for the introduction of exogenous molecules into cells are known to those of skill in the art and include, but are not limited to, protoplast transformation, silicon carbide (e.g., WHISKERS™) Agrobacterium-mediated transformation, lipid-mediated transfer (i.e., liposomes, including neutral and cationic lipids), electroporation, direct injection, cell fusion, particle bombardment (e.g., using a “gene gun”), calcium phosphate co-precipitation, DEAE-dextran-mediated transfer and viral vector-mediated transfer.

By contrast, an “endogenous” molecule is one that is normally present in a particular cell at a particular develop-mental stage under particular environmental conditions. For example, an endogenous nucleic acid can comprise a chromosome, the genome of a mitochondrion, chloroplast or other organelle, or a naturally-occurring episomal nucleic acid. Additional endogenous molecules can include proteins, for example, transcription factors and enzymes.

As used herein, the term “product of an exogenous nucleic acid” includes both polynucleotide and polypeptide products, for example, transcription products (polynucleotides such as RNA) and translation products (polypeptides).

A transgenic “event” is produced by transformation of plant cells with heterologous DNA, i.e., a nucleic acid construct that includes a transgene of interest, regeneration of a population of plants resulting from the insertion of the transgene into the genome of the plant, and selection of a particular plant characterized by insertion into a particular genome location. Transgenic progeny having the same nucleus with either heterozygous or homozygous chromosomes for the recombinant DNA are said to represent the same transgenic event. Once a transgene for a trait has been introduced into a plant, that gene can be introduced into any plant sexually compatible with the first plant by crossing, without the need for directly transforming the second plant. The heterologous DNA and flanking genomic sequence adjacent to the inserted DNA will be transferred to progeny when the event is used in a breeding program and the enhanced trait resulting from incorporation of the heterologous DNA into the plant genome will be maintained in progeny that receive the heterologous DNA.

The term “event” also refers to the presence of DNA from the original transformant, comprising the inserted DNA and flanking genomic sequence immediately adjacent to the inserted DNA, in a progeny that receives inserted DNA including the transgene of interest as the result of a sexual cross of one parental line that includes the inserted DNA (e.g., the original transformant and progeny resulting from selfing) and a parental line that does not contain the inserted DNA. The term “progeny” denotes the offspring of any generation of a parent plant prepared in accordance with the present invention. A transgenic “event” may thus be of any generation. The term “event” refers to the original transformant and progeny of the transformant that include the heterologous DNA. The term “event” also refers to progeny produced by a sexual outcross between the transformant and another variety that include the heterologous DNA. Even after repeated back crossing to a recurrent parent, the inserted DNA and flanking DNA from the transformed parent is present in the progeny of the cross at the same chromosomal location.

A “fusion” molecule is a molecule in which two or more subunit molecules are linked, preferably covalently. The subunit molecules can be the same chemical type of molecule, or can be different chemical types of molecules. Examples of the first type of fusion molecule include, but are not limited to, fusion proteins, for example, a fusion between a DNA-binding domain (e.g., ZFP, TALE and/or meganuclease DNA-binding domains) and a nuclease (cleavage) domain (e.g., endonuclease, meganuclease, etc. and fusion nucleic acids (for example, a nucleic acid encoding the fusion protein described herein). Examples of the second type of fusion molecule include, but are not limited to, a fusion between a triplex-forming nucleic acid and a polypeptide, and a fusion between a minor groove binder and a nucleic acid.

Expression of a fusion protein in a cell can result from delivery of the fusion protein to the cell or by delivery of a polynucleotide encoding the fusion protein to a cell, wherein the polynucleotide is transcribed, and the transcript is translated, to generate the fusion protein. Trans-splicing, polypeptide cleavage and polypeptide ligation can also be involved in expression of a protein in a cell. Methods for polynucleotide and polypeptide delivery to cells are presented elsewhere in this disclosure.

A “gene,” for the purposes of the present disclosure, includes a DNA region encoding a gene product (see infra), as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

“Gene expression” refers to the conversion of the information, contained in a gene, into a gene product. A gene product can be the direct transcriptional product of a gene (e.g., mRNA, tRNA, rRNA, antisense RNA, ribozyme, structural RNA or any other type of RNA) or a protein produced by translation of a mRNA. Gene products also include RNAs which are modified, by processes such as capping, polyadenylation, methylation, and editing, and proteins modified by, for example, methylation, acetylation, phosphorylation, ubiquitination, ADP-ribosylation, myristilation, and glycosylation.

“Modulation” of gene expression refers to a change in the activity of a gene. Modulation of expression can include, but is not limited to, gene activation and gene repression.

“Herbicide resistance”, and “herbicide tolerance” are intended to mean an improved capacity of a particular plant to withstand the various degrees of herbicidally induced injury that typically result in wild-type plants of a similar genotype at the same herbicidal dose. As is recognized by those skilled in the art, a plant may still be considered “resistant” even though some degree of plant injury from herbicidal exposure is apparent.

As used herein, “gene editing,” “gene edited” “genetically edited” and “gene editing effectors” refer to the use of naturally occurring or artificially engineered nucleases, also referred to as “molecular scissors.” The nucleases create specific double-stranded break (DSBs) at desired locations in the genome, which in some cases harnesses the cell's endogenous mechanisms to repair the induced break by natural processes of homologous recombination (HR) and/or nonhomologous end-joining (NHEJ). Gene editing effectors include Zinc Finger Nucleases (ZFNs), Transcription Activator-Like Effector Nucleases (TALENs), the Clustered Regularly Interspaced Short Palindromic Repeats/CAS9 (CRISPR/Cas9) system, and meganuclease re-engineered as homing endonucleases. The terms also include the use of transgenic procedures and techniques, including, for example, where the change is relatively small and/or does not introduce DNA from a foreign species. The terms “genetic manipulation” and “genetically manipulated” include gene editing techniques, as well as and/or in addition to other techniques and processes that alter or modify the nucleotide sequence of a gene or gene, or modify or alter the expression of a gene or genes.

As used herein “homing DNA technology” or “homing technology” covers any mechanisms that allow a specified molecule to be targeted to a specified DNA sequence including Zinc Finger (ZF) proteins, Transcription Activator-Like Effectors (TALEs) meganucleases, and the CRISPR/Cas9 system.

A “transgenic selectable marker” refers to an exogenous sequence comprising a marker gene operably linked to a promoter and 3′-UTR to comprise a chimeric gene expression cassette. Non-limiting examples of transgenic selectable markers include herbicide tolerance, antibiotic resistance, and visual reporter markers. The transgenic selectable marker can be integrated along with a donor sequence via targeted integration. As such, the transgenic selectable marker expresses a product that is used to assess integration of the donor. In contrast, the methods and compositions described herein allow for integration of any donor sequence without the need for co-integration of a transgenic selectable marker, for example by using a donor which mutates the endogenous gene into which it is integrated to produce a selectable marker (i.e., the selectable marker as used in this instance is not transgenic) from the endogenous target locus. Non-limiting examples of selectable markers include herbicide tolerance markers, including a mutated ALS gene as described herein.

The terms “operative linkage” and “operatively linked” (or “operably linked”) are used interchangeably with reference to a juxtaposition of two or more components (such as sequence elements), in which the components are arranged such that both components function normally and allow the possibility that at least one of the components can mediate a function that is exerted upon at least one of the other components. By way of illustration, a transcriptional regulatory sequence, such as a promoter, is operatively linked to a coding sequence if the transcriptional regulatory sequence controls the level of transcription of the coding sequence in response to the presence or absence of one or more transcriptional regulatory factors. A transcriptional regulatory sequence is generally operatively linked in cis with a coding sequence, but need not be directly adjacent to it. For example, an enhancer is a transcriptional regulatory sequence that is operatively linked to a coding sequence, even though they are not contiguous.

With respect to fusion polypeptides, the term “operatively linked” can refer to the fact that each of the components performs the same function in linkage to the other component as it would if it were not so linked. For example, with respect to a fusion polypeptide in which a DNA-binding domain (ZFP, TALE) is fused to a cleavage domain (e.g., endonuclease domain such as FokI, meganuclease domain, etc.), the DNA-binding domain and the cleavage domain are in operative linkage if, in the fusion polypeptide, the DNA-binding domain portion is able to bind its target site and/or its binding site, while the cleavage (nuclease) domain is able to cleave DNA in the vicinity of the target site. The nuclease domain may also exhibit DNA-binding capability (e.g., a nuclease fused to a ZFP or TALE domain that also can bind to DNA). Similarly, with respect to a fusion polypeptide in which a DNA-binding domain is fused to an activation or repression domain, the DNA-binding domain and the activation or repression domain are in operative link-age if, in the fusion polypeptide, the DNA-binding domain portion is able to bind its target site and/or its binding site, while the activation domain is able to upregulate gene expression or the repression domain is able to downregulate gene expression. A “functional fragment” of a protein, polypeptide or nucleic acid is a protein, polypeptide or nucleic acid whose sequence is not identical to the full-length protein, polypeptide or nucleic acid, yet retains the same function as the full-length protein, polypeptide or nucleic acid. A functional fragment can possess more, fewer, or the same number of residues as the corresponding native molecule, and/or can contain one or more amino acid or nucleotide substitutions. Methods for determining the function of a nucleic acid (e.g., coding function, ability to hybridize to another nucleic acid) are well-known in the art. Similarly, methods for determining protein function are well-known. For example, the DNA-binding function of a polypeptide can be determined, for example, by filter-binding, electrophoretic mobility-shift, or immunoprecipitation assays. DNA cleavage can be assayed by gel electrophoresis. See Ausubel et al., supra. The ability of a protein to interact with another protein can be determined, for example, by co-immunoprecipitation, two-hybrid assays or complementation, both genetic and biochemical. See, for example, Fields et al. (1989) Nature 340:245-246; U.S. Pat. No. 5,585,245 and PCT WO 98/44350.

As used herein, the terms “coding region” and “coding sequence” are used interchangeably and refer to a nucleotide sequence that encodes a polypeptide and, when placed under the control of appropriate regulatory sequences expresses the encoded polypeptide. The boundaries of a coding region are generally determined by a translation start codon at its 5′ end and a translation stop codon at its 3′ end. A “regulatory sequence” is a nucleotide sequence that regulates expression of a coding sequence to which it is operably linked. Non-limiting examples of regulatory sequences include promoters, enhancers, transcription initiation sites, translation start sites, translation stop sites, and transcription terminators.

A polynucleotide that includes a coding region may include heterologous nucleotides that flank one or both sides of the coding region. As used herein, “heterologous nucleotides” refer to nucleotides that are not normally present flanking a coding region that is present in a wild-type cell. For instance, a coding region present in a wild-type microbe and encoding a Cas9 polypeptide is flanked by homologous sequences, and any other nucleotide sequence flanking the coding region is considered to be heterologous. Examples of heterologous nucleotides include, but are not limited to regulatory sequences. Typically, heterologous nucleotides are present in a polynucleotide disclosed herein through the use of standard genetic and/or recombinant methodologies well known to one skilled in the art. A polynucleotide disclosed herein may be included in a suitable vector.

As used herein, “genetically modified cell” refers to a cell, which has been altered “by the hand of man.” A genetically modified cell, includes a cell, callus, tissue, plant, or animal into which has been introduced an exogenous polynucleotide. Genetically modified cell, also refers to a cell that has been genetically manipulated such that endogenous nucleotides have been altered to include a mutation, such as a deletion, an insertion, a transition, a transversion, or a combination thereof. For instance, an endogenous coding region could be deleted. Such mutations may result in a polypeptide having a different amino acid sequence than was encoded by the endogenous polynucleotide. Another example of a genetically modified cell, callus, tissue, plant, or animal is one having an altered regulatory sequence, such as a promoter, to result in increased or decreased expression of an operably linked endogenous coding region.

It is also to be understood that two different transgenic and/or genetically manipulated plants can be mated to produce offspring that contain two independently segregating added, exogenous genes. Selling of appropriate progeny can produce plants that are homozygous for both added, exogenous and/or modified genes. Alternatively, inbred lines containing the individual exogenous genes may be crossed to produce hybrid seed that is heterozygous for each gene, and useful for production of hybrid plants that exhibit multiple beneficial phenotypes as the result of expression of each of the exogenous genes. Descriptions of breeding methods that are commonly used for different traits and crops can be found in various references, e.g., Allard, “Principles of Plant Breeding,” John Wiley & Sons, NY, U. of CA, Davis, Calif., 50-98, 1960; Simmonds, “Principles of Crop Improvement,” Longman, Inc., NY, 369-399, 1979; Sneep and Hendriksen, “Plant Breeding Perspectives,” Wageningen (ed), Center for Agricultural Publishing and Documentation, 1979.

Transgenic plants comprising or derived from plant cells of this invention transformed with recombinant DNA can be further enhanced with “stacked” traits, e.g. a crop plant having an enhanced trait resulting from expression of DNA disclosed herein in combination with herbicide and/or pest resistance traits. For example, genes of the current invention can be stacked with other traits of agronomic interest, such as a trait providing herbicide resistance, or insect resistance, such as using a gene from Bacillus thuringiensis to provide resistance against lepidopteran, coliopteran, homopteran, hemiopteran, and other insects.

Herbicides for which transgenic plant tolerance has been demonstrated and the method of the present invention can be applied include, but are not limited to, glyphosate, dicamba, glufosinate, sulfonylurea, bromoxynil and norflurazon herbicides. Polynucleotide molecules encoding proteins involved in herbicide tolerance are well-known in the art and include, but are not limited to, a polynucleotide molecule encoding 5-enolpyruvylshikimate-3-phosphate synthase (EPSPS) disclosed in U.S. Pat. Nos. 5,094,945; 5,627,061; 5,633,435 and 6,040,497 for imparting glyphosate tolerance; polynucleotide molecules encoding a glyphosate oxidoreductase (GOX) disclosed in U.S. Pat. No. 5,463,175; and a glyphosate-N-acetyl transferase (GAT) disclosed in US Patent Application Publication 2003/0083480 A1 also for imparting glyphosate tolerance; dicamba monooxygenase disclosed in US Patent Application Publication 2003/0135879 A1 for imparting dicamba tolerance; a polynucleotide molecule encoding bromoxynil nitrilase (Bxn) disclosed in U.S. Pat. No. 4,810,648 for imparting bromoxynil tolerance; a polynucleotide molecule encoding phytoene desaturase (crtI) described in Misawa et al., (1993) Plant J. 4:833-840 and in Misawa et al., (1994) Plant J. 6:481-489 for norflurazon tolerance; a polynucleotide molecule encoding acetohydroxyacid synthase (AHAS, also known as, ALS) described in Sathasiivan et al. (1990) Nucl. Acids Res. 18:2188-2193 for imparting tolerance to sulfonylurea herbicides; polynucleotide molecules known as bar genes disclosed in DeBlock, et al. (1987) EMBO J. 6:2513-2519 for imparting glufosinate and bialaphos tolerance; polynucleotide molecules disclosed in US Patent Application Publication 2003/010609 A1 for imparting N-amino methyl phosphonic acid tolerance; polynucleotide molecules disclosed in U.S. Pat. No. 6,107,549 for imparting pyridine herbicide resistance; molecules and methods for imparting tolerance to multiple herbicides such as glyphosate, atrazine, ALS inhibitors, isoxoflutole and glufosinate herbicides are disclosed in U.S. Pat. No. 6,376,754 and US Patent Application Publication 2002/0112260. Molecules and methods for imparting insect/nematode/virus resistance are disclosed in U.S. Pat. Nos. 5,250,515; 5,880,275; 6,506,599; 5,986,175 and US Patent Application Publication 2003/0150017 A1.

### Transformation

Numerous methods for plant transformation have been developed, including biological and physical, plant transformation protocols. See, for example, Miki et al., “Procedures for Introducing Foreign DNA into Plants” in Methods in Plant Molecular Biology and Biotechnology, Glick, B. R. and Thompson, J. E. Eds. (CRC Press, Inc., Boca Raton, 1993) pages 67-88. In addition, expression vectors and in vitro culture methods for plant cell or tissue transformation and regeneration of plants are available. See, for example, Gruber et al., “Vectors for Plant Transformation” in Methods in Plant Molecular Biology and Biotechnology, Glick, B. R. and Thompson, J. E. Eds. (CRC Press, Inc., Boca Raton, 1993) pages 89-119.

A. Agrobacterium-Mediated Transformation

One method for introducing an expression vector into plants is based on the natural transformation system of Agrobacterium. See, for example, Horsch et al., Science 227: 1229 (1985). A. tumefaciens and A. rhizogenes are plant pathogenic soil bacteria which genetically transform plant cells. The Ti and Ri plasmids of A. tumefaciens and A. rhizogenes, respectively, carry genes responsible for genetic transformation of the plant. See, for example, Kado, C. I., Crit. Rev. Plant. Sci. 10: 1 (1991). Descriptions of Agrobacterium vector systems and methods for Agrobacterium-mediated gene transfer are provided by Gruber et al., supra, Miki et al., supra, and Moloney et al., Plant Cell Reports 8: 238 (1989). See also, U.S. Pat. No. 5,563,055, (Townsend and Thomas), issued Oct. 8, 1996.

B. Direct Gene Transfer

Several methods of plant transformation, collectively referred to as direct gene transfer, have been developed as an alternative to Agrobacterium-mediated transformation. A generally applicable method of plant transformation is microprojectile-mediated transformation wherein DNA is carried on the surface of microprojectiles measuring 1 to 4 □m. The expression vector is introduced into plant tissues with a biolistic device that accelerates the microprojectiles to speeds of 300 to 600 m/s which is sufficient to penetrate plant cell walls and membranes. Sanford et al., Part. Sci. Technol. 5: 27 (1987), Sanford, J. C., Trends Biotech. 6: 299 (1988), Klein et al., Bio/Technology 6: 559-563 (1988), Sanford, J. C., Physiol Plant 79: 206 (1990), Klein et al., Biotechnology 10: 268 (1992). See also U.S. Pat. No. 5,015,580 (Christou, et al), issued May 14, 1991; U.S. Pat. No. 5,322,783 (Tomes, et al.), issued Jun. 21, 1994.

Another method for physical delivery of DNA to plants is sonication of target cells. Zhang et al., Bio/Technology 9: 996 (1991). Alternatively, liposome or spheroplast fusion have been used to introduce expression vectors into plants. Deshayes et al., EMBO J., 4: 2731 (1985), Christou et al., Proc Natl. Acad. Sci. U.S.A. 84: 3962 (1987). Direct uptake of DNA into protoplasts using CaCl₂ precipitation, polyvinyl alcohol or poly-L-ornithine have also been reported. Hain et al., Mol. Gen. Genet. 199: 161 (1985) and Draper et al., Plant Cell Physiol. 23: 451 (1982). Electroporation of protoplasts and whole cells and tissues have also been described. Donn et al., In Abstracts of VIIth International Congress on Plant Cell and Tissue Culture IAPTC, A2-38, p 53 (1990); D'Halluin et al., Plant Cell 4: 1495-1505 (1992) and Spencer et al., Plant Mol. Biol. 24: 51-61 (1994).

Following transformation of target tissues, expression of the above-described selectable marker genes allows for preferential selection of transformed cells, tissues and/or plants, using regeneration and selection methods now well known in the art.

It is often desirable to have the DNA sequence in homozygous state which may require more than one transformation event to create a parental line, requiring transformation with a first and second recombinant DNA molecule both of which encode the same gene product. It is further contemplated in some of the embodiments of the process of the invention that a plant cell be transformed with a recombinant DNA molecule containing at least two DNA sequences or be transformed with more than one recombinant DNA molecule. The DNA sequences or recombinant DNA molecules in such embodiments may be physically linked, by being in the same vector, or physically separate on different vectors. A cell may be simultaneously transformed with more than one vector provided that each vector has a unique selection marker gene. Alternatively, a cell may be transformed with more than one vector sequentially allowing an intermediate regeneration step after transformation with the first vector. Further, it may be possible to perform a sexual cross between individual plants or plant lines containing different DNA sequences or recombinant DNA molecules preferably the DNA sequences or the recombinant molecules are linked or located on the same chromosome, and then selecting from the progeny of the cross, plants containing both DNA sequences or recombinant DNA molecules.

Expression of recombinant DNA molecules containing the DNA sequences and promoters described herein in transformed plant cells may be monitored using Northern blot techniques and/or Southern blot techniques known to those of skill in the art.

The transformed cells may then be regenerated into a transgenic plant. The regenerated plants are transferred to standard soil conditions and cultivated in a conventional manner.

After the expression or inhibition cassette is stably incorporated into regenerated transgenic plants, it can be transferred to other plants by sexual crossing. Any of a number of standard breeding techniques can be used, depending upon the species to be crossed.

It may be useful to generate a number of individual transformed plants with any recombinant construct in order to recover plants free from any position effects. It may also be preferable to select plants that contain more than one copy of the introduced recombinant DNA molecule such that high levels of expression of the recombinant molecule are obtained.

As indicated above, it may be desirable to produce plant lines which are homozygous for a particular gene. In some species this is accomplished rather easily by the use of another culture or isolated microspore culture. This is especially true for the oil seed crop Brassica napus (Keller and Armstrong, Z. flanzenzucht 80:100-108, 1978). By using these techniques, it is possible to produce a haploid line that carries the inserted gene and then to double the chromosome number either spontaneously or by the use of colchicine. This gives rise to a plant that is homozygous for the inserted gene, which can be easily assayed for if the inserted gene carries with it a suitable selection marker gene for detection of plants carrying that gene. Alternatively, plants may be self-fertilized, leading to the production of a mixture of seed that consists of, in the simplest case, three types, homozygous (25%), heterozygous (50%) and null (25%) for the inserted gene. Although it is relatively easy to score null plants from those that contain the gene, it is possible in practice to score the homozygous from heterozygous plants by southern blot analysis in which careful attention is paid to the loading of exactly equivalent amounts of DNA from the mixed population, and scoring heterozygotes by the intensity of the signal from a probe specific for the inserted gene. It is advisable to verify the results of the southern blot analysis by allowing each independent transformant to self-fertilize, since additional evidence for homozygosity can be obtained by the simple fact that if the plant was homozygous for the inserted gene, all of the subsequent plants from the selfed seed will contain the gene, while if the plant was heterozygous for the gene, the generation grown from the selfed seed will contain null plants. Therefore, with simple selfing one can easily select homozygous plant lines that can also be confirmed by southern blot analysis.

Creation of homozygous parental lines makes possible the production of hybrid plants and seeds which will contain a modified protein component. Transgenic homozygous parental lines are maintained with each parent containing either the first or second recombinant DNA sequence operably linked to a promoter. Also incorporated in this scheme are the advantages of growing a hybrid crop, including the combining of more valuable traits and hybrid vigor.

The nucleotide constructs of the invention also encompass nucleotide constructs that may be employed in methods for altering or mutating a genomic nucleotide sequence in an organism, including, but not limited to, chimeric vectors, chimeric mutational vectors, chimeric repair vectors, mixed-duplex oligonucleotides, self-complementary chimeric oligonucleotides, and recombinogenic oligonucleobases. Such nucleotide constructs and methods of use, such as, for example, chimeraplasty, are known in the art. Chimeraplasty involves the use of such nucleotide constructs to introduce site-specific changes into the sequence of genomic DNA within an organism. See, U.S. Pat. Nos. 5,565,350; 5,731,181; 5,756,325; 5,760,012; 5,795,972; and 5,871,984; all of which are herein incorporated by reference. See also, WO 98/49350, WO 99/07865, WO 99/25821, and Beetham et al. (1999) Proc. Natl. Acad. Sci. USA 96:8774-8778; herein incorporated by reference.

### Marker Genes

Recombinant DNA molecules containing any of the DNA sequences and promoters described herein may additionally contain selection marker genes which encode a selection gene product which confer on a plant cell resistance to a chemical agent or physiological stress, or confers a distinguishable phenotypic characteristic to the cells such that plant cells transformed with the recombinant DNA molecule may be easily selected using a selective agent. One such selection marker gene is neomycin phosphotransferase (NPT II) which confers resistance to kanamycin and the antibiotic G-418. Cells transformed with this selection marker gene may be selected for by assaying for the presence in vitro of phosphorylation of kanamycin using techniques described in the literature or by testing for the presence of the mRNA coding for the NPT II gene by Northern blot analysis in RNA from the tissue of the transformed plant. Polymerase chain reactions are also used to identify the presence of a transgene or expression using reverse transcriptase PCR amplification to monitor expression and PCR on genomic DNA. Other commonly used selection markers include the ampicillin resistance gene, the tetracycline resistance, and the hygromycin resistance gene. Another such selection marker is the expression of fluorescent proteins within the transformed plant cells (e.g., GFP, CFP, YFP, etc.). Transformed plant cells thus selected can be induced to differentiate into plant structures which will eventually yield whole plants. It is to be understood that a selection marker gene may also be native to a plant.

### Integration of a Heterologous Nucleic Acid Insert

Site-specific integration of an exogenous nucleic acid at a native locus may be accomplished by any technique known to those of skill in the art. In some embodiments, integration of a heterologous nucleic acid insert at a native dandelion locus comprises contacting a cell (e.g., an isolated cell or a cell in a tissue) with a nucleic acid molecule comprising the heterologous nucleic acid insert. In examples, such a nucleic acid molecule may comprise nucleotide sequences flanking the exogenous nucleic acid that facilitate homologous recombination between the nucleic acid molecule and at least one native locus. In particular examples, the nucleotide sequences flanking the exogenous nucleic acid that facilitate homologous recombination may be complementary to endogenous nucleotides of the native locus. In some embodiments, the heterologous nucleic acid insert provides for improved herbicide tolerance, for example, herbicide resistance genes, including, but not limited to glyphosphate-, ALS- (imidazoline, sulfonylurea), aryloxyalkanoate-, and HPPD-, PPO-, and glufosinate-resistance genes. In some embodiments, a plurality of exogenous nucleic acids may be integrated, such as in gene stacking.

Integration of a nucleic acid may be facilitated (e.g., catalyzed) in some embodiments by endogenous cellular machinery of a host cell, such as, for example and without limitation, endogenous DNA and endogenous recombinase enzymes. In some embodiments, integration of a nucleic acid may be facilitated by one or more factors (e.g., polypeptides) that are provided to a host cell. For example, nuclease(s), recombinase(s), and/or ligase polypeptides may be provided (either independently or as part of a chimeric polypeptide) by contacting the polypeptides with the host cell, or by expressing the polypeptides within the host cell. Accordingly, in some examples, a nucleic acid comprising a nucleotide sequence encoding at least one nuclease, recombinase, and/or ligase polypeptide may be introduced into the host cell, either concurrently or sequentially with a nucleic acid to be integrated site-specifically, wherein the at least one nuclease, recombinase, and/or ligase polypeptide is expressed from the nucleotide sequence in the host cell.

### Homology Directed Repair (HDR)

Homology directed repair (HDR) is a mechanism in cells to repair ssDNA and double stranded DNA (dsDNA) lesions. This repair mechanism can be used by the cell when there is an HDR template present that has a sequence with significant homology to the lesion site.

In one embodiment of the invention, genetically modified herbicide-resistant dandelions exploit known mutations in an endogenous gene such as known mutations in the ALS gene (acetolactate synthase (ALS), also known as acetohydroxyacid synthase (AHAS)) that confer tolerance to Group B herbicides, or ALS inhibitor herbicides such as imidazolinone or sulfonylurea. Sulfonylurea herbicides prevent branched amino acid biosynthesis in plants because of the inhibition of the enzyme acetolactate synthase (ALS). Resistance to these herbicides have been demonstrated as a result of single-amino acid changes in the ALS protein at position 197 in Arabidopsis (Pro to Ser) and tobacco (Pro to Gln or Ala) and at a corresponding location, position 178, in soybean (Pro to Ser). It is an aspect of the invention to utilize corresponding and functionally similar mutations in dandelion plants employing RNA-guided Cas9 or TALENs to facilitate specific DNA changes in a native Taraxacum gene. In a non-limiting example, gRNA-expressing DNA vectors targeting different sites within the ALS genes are generated. gRNAs can be specific to a single ALS allele or capable of targeting all ALS genes within a dandelion genome with approximately the same efficiency and result in stable event recovery. To generate ALS-edited alleles, a fragment of homology is cloned into a plasmid vector, and single-stranded DNA oligos generated as repair templates. The repair templates contain several nucleotide changes compared to the native sequence. Specifically, in the exemplary method the repair template includes a single-nucleotide change that directs editing of DNA sequences corresponding a Pro to a Ser or Ala.

The HDR template is a nucleic acid that comprises the allele that is being introgressed. The template may be a dsDNA or a single-stranded DNA (ssDNA). ssDNA templates are preferably from about 20 to about 5000 residues although other lengths can be used. Artisans will immediately appreciate that all ranges and values within the explicitly stated range are contemplated; e.g., from 500 to 1500 residues, from 20 to 100 residues, and so forth. The template may further comprise flanking sequences that provide homology to DNA adjacent to the endogenous allele or the DNA that is to be replaced. The template may also comprise a sequence that is bound to a targeted nuclease system, and is thus the cognate binding site for the system's DNA-binding member. The term cognate refers to two biomolecules that typically interact, for example, a receptor and its ligand. In the context of HDR processes, one of the biomolecules may be designed with a sequence to bind with an intended, i.e., cognate, DNA site or protein site.

### Targeted Endonuclease Systems

Genome editing tools such as transcription activator-like effector nucleases (TALENs) and zinc finger nucleases (ZFNs) have impacted the fields of biotechnology, gene therapy and functional genomic studies in many organisms. More recently, RNA-guided endonucleases (RGENs) are directed to their target sites by a complementary RNA molecule. The Cas9/CRISPR system is a REGEN. tracrRNA is another such tool. These are examples of targeted nuclease systems: these system have a DNA-binding member that localizes the nuclease to a target site. The site is then cut by the nuclease. TALENs and ZFNs have the nuclease fused to the DNA-binding member. Cas9/CRISPR are cognates that find each other on the target DNA. The DNA-binding member has a cognate sequence in the chromosomal DNA. The DNA-binding member is typically designed in light of the intended cognate sequence so as to obtain a nucleolytic action at nor near an intended site. Certain embodiments are applicable to all such systems without limitation; including, embodiments that minimize nuclease re-cleavage, embodiments for making SNPs with precision at an intended residue, and placement of the allele that is being introgressed at the DNA-binding site.

### DNA-Binding Polypeptides

In some embodiments, site-specific integration may be accomplished by utilizing factors that are capable of recognizing and binding to particular nucleotide sequences, for example, in the genome of a host organism. For instance, many proteins comprise polypeptide domains that are capable of recognizing and binding to DNA in a site-specific manner. A DNA sequence that is recognized by a DNA-binding polypeptide may be referred to as a “target” sequence. Polypeptide domains that are capable of recognizing and binding to DNA in a site-specific manner generally fold correctly and function independently to bind DNA in a site-specific manner, even when expressed in a polypeptide other than the protein from which the domain was originally isolated. Similarly, target sequences for recognition and binding by DNA-binding polypeptides are generally able to be recognized and bound by such polypeptides, even when present in large DNA structures (e.g., a chromosome), particularly when the site where the target sequence is located is one known to be accessible to soluble cellular proteins (e.g., a gene).

While DNA-binding polypeptides identified from proteins that exist in nature typically bind to a discrete nucleotide sequence or motif (e.g., a consensus recognition sequence), methods exist and are known in the art for modifying many such DNA-binding polypeptides to recognize a different nucleotide sequence or motif. DNA-binding polypeptides include, for example and without limitation: zinc finger DNA-binding domains; leucine zippers; UPA DNA-binding domains; GAL4; TAL; LexA; a Tet repressor; LacR; and a steroid hormone receptor.

In some examples, a DNA-binding polypeptide is a zinc finger. Individual zinc finger motifs can be designed to target and bind specifically to any of a large range of DNA sites. Canonical Cys₂His₂ (as well as non-canonical Cys₃His) zinc finger polypeptides bind DNA by inserting an .alpha.-helix into the major groove of the target DNA double helix. Recognition of DNA by a zinc finger is modular; each finger contacts primarily three consecutive base pairs in the target, and a few key residues in the polypeptide mediate recognition. By including multiple zinc finger DNA-binding domains in a targeting endonuclease, the DNA-binding specificity of the targeting endonuclease may be further increased (and hence the specificity of any gene regulatory effects conferred thereby may also be increased). See, e.g., Urnov et al. (2005) Nature 435:646-51. Thus, one or more zinc finger DNA-binding polypeptides may be engineered and utilized such that a targeting endonuclease introduced into a host cell interacts with a DNA sequence that is unique within the genome of the host cell.

Preferably, the zinc finger protein is non-naturally occurring in that it is engineered to bind to a target site of choice. See, for example, See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416; U.S. Pat. Nos. 6,453,242; 6,534,261; 6,599,692; 6,503,717; 6,689,558; 7,030,215; 6,794,136; 7,067,317; 7,262,054; 7,070,934; 7,361,635; 7,253,273; and U.S. Patent Publication Nos. 2005/0064474; 2007/0218528; 2005/0267061, all incorporated herein by reference in their entireties.

An engineered zinc finger binding domain can have a novel binding specificity, compared to a naturally-occurring zinc finger protein. Engineering methods include, but are not limited to, rational design and various types of selection. Rational design includes, for example, using databases comprising triplet (or quadruplet) nucleotide sequences and individual zinc finger amino acid sequences, in which each triplet or quadruplet nucleotide sequence is associated with one or more amino acid sequences of zinc fingers which bind the particular triplet or quadruplet sequence. See, for example, co-owned U.S. Pat. Nos. 6,453,242 and 6,534,261, incorporated by reference herein in their entireties.

Exemplary selection methods, including phage display and two-hybrid systems, are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237. In addition, enhancement of binding specificity for zinc finger binding domains has been described, for example, in co-owned WO 02/077227.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein.

Selection of target sites; ZFPs and methods for design and construction of fusion proteins (and polynucleotides encoding same) are known to those of skill in the art and described in detail in U.S. Pat. Nos. 6,140,0815; 789,538; 6,453,242; 6,534,261; 5,925,523; 6,007,988; 6,013,453; 6,200,759; WO 95/19431; WO 96/06166; WO 98/53057; WO 98/54311; WO 00/27878; WO 01/60970 WO 01/88197; WO 02/099084; WO 98/53058; WO 98/53059; WO 98/53060; WO 02/016536 and WO 03/016496.

In addition, as disclosed in these and other references, zinc finger domains and/or multi-fingered zinc finger proteins may be linked together using any suitable linker sequences, including for example, linkers of 5 or more amino acids in length. See, also, U.S. Pat. Nos. 6,479,626; 6,903,185; and 7,153,949 for exemplary linker sequences 6 or more amino acids in length. The proteins described herein may include any combination of suitable linkers between the individual zinc fingers of the protein.

In some examples, a DNA-binding polypeptide is a DNA-binding domain from GAL4. GAL4 is a modular transactivator in Saccharomyces cerevisiae, but it also operates as a transactivator in many other organisms. See, e.g., Sadowski et al. (1988) Nature 335:563-4. In this regulatory system, the expression of genes encoding enzymes of the galactose metabolic pathway in S. cerevisiae is stringently regulated by the available carbon source. Johnston (1987) Microbiol. Rev. 51:458-76. Transcriptional control of these metabolic enzymes is mediated by the interaction between the positive regulatory protein, GAL4, and a 17 bp symmetrical DNA sequence to which GAL4 specifically binds (the UAS).

Native GAL4 consists of 881 amino acid residues, with a molecular weight of 99 kDa. GAL4 comprises functionally autonomous domains, the combined activities of which account for activity of GAL4 in vivo. Ma and Ptashne (1987) Cell 48:847-53); Brent and Ptashne (1985) Cell 43(3 Pt 2):729-36. The N-terminal 65 amino acids of GAL4 comprise the GAL4 DNA-binding domain. Keegan et al. (1986) Science 231:699-704; Johnston (1987) Nature 328:353-5. Sequence-specific binding requires the presence of a divalent cation coordinated by 6 Cys residues present in the DNA binding domain. The coordinated cation-containing domain interacts with and recognizes a conserved CCG triplet at each end of the 17 bp UAS via direct contacts with the major groove of the DNA helix. Marmorstein et al. (1992) Nature 356:408-14. The DNA-binding function of the protein positions C-terminal transcriptional activating domains in the vicinity of the promoter, such that the activating domains can direct transcription.

Additional DNA-binding polypeptides that may be utilized in certain embodiments include, for example and without limitation, a binding sequence from a AVRBS3-inducible gene; a consensus binding sequence from a AVRBS3-inducible gene or synthetic binding sequence engineered therefrom (e.g., UPA DNA-binding domain); TAL; LexA (see, e.g., Brent & Ptashne (1985), supra); LacR (see, e.g., Labow et al. (1990) Mol. Cell. Biol. 10:3343-56; Baim et al. (1991) Proc. Natl. Acad. Sci. USA 88(12):5072-6); a steroid hormone receptor (Ellliston et al. (1990) J. Biol. Chem. 265:11517-121); the Tet repressor (U.S. Pat. No. 6,271,341) and a mutated Tet repressor that binds to a tet operator sequence in the presence, but not the absence, of tetracycline (Tc); the DNA-binding domain of NF-.kappa.B; and components of the regulatory system described in Wang et al. (1994) Proc. Natl. Acad. Sci. USA 91(17):8180-4, which utilizes a fusion of GAL4, a hormone receptor, and VP16.

In certain embodiments, the DNA-binding domain of one or more of the nucleases used in the methods and compositions described herein comprises a naturally occurring or engineered (non-naturally occurring) TAL effector DNA binding domain. See, e.g., U.S. Patent Publication No. 20110301073, incorporated by reference in its entirety herein.

In other embodiments, the nuclease comprises a CRISPR/Cas system. The CRISPR (clustered regularly interspaced short palindromic repeats) locus, which encodes RNA components of the system, and the Cas (CRISPR-associated) locus, which encodes proteins (Jansen et al., 2002. Mol. Microbiol. 43: 1565-1575; Makarova et al., 2002. Nucleic Acids Res. 30: 482-496; Makarova et al., 2006. Biol. Direct 1: 7; Haft et al., 2005. PLoS Comput. Biol. 1: e60) make up the gene sequences of the CRISPR/Cas nuclease system. CRISPR loci in microbial hosts contain a combination of Cas genes as well as non-coding RNA elements capable of programming the specificity of the CRISPR-mediated nucleic acid cleavage.

The Type II CRISPR is one of the most well characterized systems and carries out targeted DNA double-strand break in four sequential steps. First, two non-coding RNA, the pre-crRNA array and tracrRNA, are transcribed from the CRISPR locus. Second, tracrRNA hybridizes to the repeat regions of the pre-crRNA and mediates the processing of pre-crRNA into mature crRNAs containing individual spacer sequences. Third, the mature crRNA:tracrRNA complex directs Cas9 to the target DNA via Wastson-Crick base-pairing between the spacer on the crRNA and the protospacer on the target DNA next to the protospacer adjacent motif (PAM), an additional requirement for target recognition. Finally, Cas9 mediates cleavage of target DNA to create a double-stranded break within the protospacer. Activity of the CRISPR/Cas system comprises of three steps: (i) insertion of alien DNA sequences into the CRISPR array to prevent future attacks, in a process called ‘ adaptation’, (ii) expression of the relevant proteins, as well as expression and processing of the array, followed by (iii) RNA-mediated interference with the foreign nucleic acid. Thus, in the bacterial cell, several Cas proteins are involved with the natural function of the CRISPR/Cas system and serve roles in functions such as insertion of the foreign DNA etc.

Compositions and methods for making and using CRISPR-Cas systems are described in U.S. Pat. No. 8,697,359, entitled “CRISPR-CAS SYSTEMS AND METHODS FOR ALTERING EXPRESSION OF GENE PRODUCTS,” which is incorporated herein in its entirety.

In certain embodiments, Cas protein may be a “functional derivative” of a naturally occurring Cas protein. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative to hydrolyze a DNA substrate into fragments. The term “derivative” encompasses both amino acid sequence variants of polypeptide, covalent modifications, and fusions thereof. Suitable derivatives of a Cas polypeptide or a fragment thereof include but are not limited to mutants, fusions, covalent modifications of Cas protein or a fragment thereof. Cas protein, which includes Cas protein or a fragment thereof, as well as derivatives of Cas protein or a fragment thereof, may be obtainable from a cell or synthesized chemically or by a combination of these two procedures. The cell may be a cell that naturally produces Cas protein, or a cell that naturally produces Cas protein and is genetically engineered to produce the endogenous Cas protein at a higher expression level or to produce a Cas protein from an exogenously introduced nucleic acid, which nucleic acid encodes a Cas that is same or different from the endogenous Cas. In some case, the cell does not naturally produce Cas protein and is genetically engineered to produce a Cas protein.

In particular embodiments, a DNA-binding polypeptide specifically recognizes and binds to a target nucleotide sequence comprised within a genomic nucleic acid of a host organism. Any number of discrete instances of the target nucleotide sequence may be found in the host genome in some examples. The target nucleotide sequence may be rare within the genome of the organism (e.g., fewer than about 10, about 9, about 8, about 7, about 6, about 5, about 4, about 3, about 2, or about 1 copy(ies) of the target sequence may exist in the genome). For example, the target nucleotide sequence may be located at a unique site within the genome of the organism. Target nucleotide sequences may be, for example and without limitation, randomly dispersed throughout the genome with respect to one another; located in different linkage groups in the genome; located in the same linkage group; located on different chromosomes; located on the same chromosome; located in the genome at sites that are expressed under similar conditions in the organism (e.g., under the control of the same, or substantially functionally identical, regulatory factors); and located closely to one another in the genome (e.g., target sequences may be comprised within nucleic acids integrated as concatemers at genomic loci).

### Targeting Endonucleases

In particular embodiments, a DNA-binding polypeptide that specifically recognizes and binds to a target nucleotide sequence may be comprised within a chimeric polypeptide, so as to confer specific binding to the target sequence upon the chimeric polypeptide. In examples, such a chimeric polypeptide may comprise, for example and without limitation, nuclease, recombinase, and/or ligase polypeptides, as these polypeptides are described above. Chimeric polypeptides comprising a DNA-binding polypeptide and a nuclease, recombinase, and/or ligase polypeptide may also comprise other functional polypeptide motifs and/or domains, such as for example and without limitation: a spacer sequence positioned between the functional polypeptides in the chimeric protein; a leader peptide; a peptide that targets the fusion protein to an organelle (e.g., the nucleus); polypeptides that are cleaved by a cellular enzyme; peptide tags (e.g., Myc, His, etc.); and other amino acid sequences that do not interfere with the function of the chimeric polypeptide.

Functional polypeptides (e.g., DNA-binding polypeptides and nuclease polypeptides) in a chimeric polypeptide may be operatively linked. In some embodiments, functional polypeptides of a chimeric polypeptide may be operatively linked by their expression from a single polynucleotide encoding at least the functional polypeptides ligated to each other in-frame, so as to create a chimeric gene encoding a chimeric protein. In alternative embodiments, the functional polypeptides of a chimeric polypeptide may be operatively linked by other means, such as by cross-linkage of independently expressed polypeptides.

In some embodiments, a DNA-binding polypeptide, or guide RNA that specifically recognizes and binds to a target nucleotide sequence may be comprised within a natural isolated protein (or mutant thereof), wherein the natural isolated protein or mutant thereof also comprises a nuclease polypeptide (and may also comprise a recombinase and/or ligase polypeptide). Examples of such isolated proteins include TALENs, recombinases (e.g., Cre, Hin, Tre, and FLP recombinase), RNA-guided CRISPR/Cas9, and meganucleases.

As used herein, the term “targeting endonuclease” refers to natural or engineered isolated proteins and mutants thereof that comprise a DNA-binding polypeptide or guide RNA and a nuclease polypeptide, as well as to chimeric polypeptides comprising a DNA-binding polypeptide or guide RNA and a nuclease. Any targeting endonuclease comprising a DNA-binding polypeptide or guide RNA that specifically recognizes and binds to a target nucleotide sequence comprised within a GOI (e.g., either because the target sequence is comprised within the native sequence at the locus, or because the target sequence has been introduced into the locus, for example, by recombination) may be utilized in certain embodiments.

Some examples of chimeric polypeptides that may be useful in particular embodiments of the invention include, without limitation, combinations of the following polypeptides: zinc finger DNA-binding polypeptides; a FokI nuclease polypeptide; TALE domains; leucine zippers; transcription factor DNA-binding motifs; and DNA recognition and/or cleavage domains isolated from, for example and without limitation, a TALEN, a recombinase (e.g., Cre, Hin, RecA, Tre, and FLP recombinases), RNA-guided CRISPR-Cas9, a meganuclease; and others known to those in the art. Particular examples include a chimeric protein comprising a site-specific DNA binding polypeptide and a nuclease polypeptide. Chimeric polypeptides may be engineered by methods known to those of skill in the art to alter the recognition sequence of a DNA-binding polypeptide comprised within the chimeric polypeptide, so as to target the chimeric polypeptide to a particular nucleotide sequence of interest.

In certain embodiments, the chimeric polypeptide comprises a DNA-binding domain (e.g., zinc finger, TAL-effector domain, etc.) and a nuclease (cleavage) domain. The cleavage domain may be heterologous to the DNA-binding domain, for example a zinc finger DNA-binding domain and a cleavage domain from a nuclease or a TALEN DNA-binding domain and a cleavage domain, or meganuclease DNA-binding domain and cleavage domain from a different nuclease. Heterologous cleavage domains can be obtained from any endonuclease or exonuclease. Exemplary endonucleases from which a cleavage domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., 51 Nuclease; mung bean nuclease; pancreatic DNase I; micrococcal nuclease; yeast HO endonuclease; see also Linn et al. (eds.) Nucleases, Cold Spring Harbor Laboratory Press, 1993). One or more of these enzymes (or functional fragments thereof) can be used as a source of cleavage domains and cleavage half-domains.

Similarly, a cleavage half-domain can be derived from any nuclease or portion thereof, as set forth above, that requires dimerization for cleavage activity. In general, two fusion proteins are required for cleavage if the fusion proteins comprise cleavage half-domains. Alternatively, a single protein comprising two cleavage half-domains can be used. The two cleavage half-domains can be derived from the same endonuclease (or functional fragments thereof), or each cleavage half-domain can be derived from a different endonuclease (or functional fragments thereof). In addition, the target sites for the two fusion proteins are preferably disposed, with respect to each other, such that binding of the two fusion proteins to their respective target sites places the cleavage half-domains in a spatial orientation to each other that allows the cleavage half-domains to form a functional cleavage domain, e.g., by dimerizing. Thus, in certain embodiments, the near edges of the target sites are separated by 5-8 nucleotides or by 15-18 nucleotides. However any integral number of nucleotides, or nucleotide pairs, can intervene between two target sites (e.g., from 2 to 50 nucleotide pairs or more). In general, the site of cleavage lies between the target sites.

Restriction endonucleases (restriction enzymes) are present in many species and are capable of sequence-specific binding to DNA (at a recognition site), and cleaving DNA at or near the site of binding, for example, such that one or more exogenous sequences (donors/trangsenes) are integrated at or near the binding (target) sites. Certain restriction enzymes (e.g., Type IIS) cleave DNA at sites removed from the recognition site and have separable binding and cleavage domains. For example, the Type IIS enzyme Fok I catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982. Thus, in one embodiment, fusion proteins comprise the cleavage domain (or cleavage half-domain) from at least one Type IIS restriction enzyme and one or more zinc finger binding domains, which may or may not be engineered.

An exemplary Type IIS restriction enzyme, whose cleavage domain is separable from the binding domain, is Fok I. This particular enzyme is active as a dimer. Bitinaite et al. (1998) Proc. Natl. Acad. Sci. USA 95: 10,570-10,575. Accordingly, for the purposes of the present disclosure, the portion of the Fok I enzyme used in the disclosed fusion proteins is considered a cleavage half-domain. Thus, for targeted double-stranded cleavage and/or targeted replacement of cellular sequences using zinc finger-Fok I fusions, two fusion proteins, each comprising a FokI cleavage half-domain, can be used to reconstitute a catalytically active cleavage domain. Alternatively, a single polypeptide molecule containing a DNA binding domain and two Fok I cleavage half-domains can also be used.

A cleavage domain or cleavage half-domain can be any portion of a protein that retains cleavage activity, or that retains the ability to multimerize (e.g., dimerize) to form a functional cleavage domain.

Exemplary Type IIS restriction enzymes are described in U.S. Patent Publication No. 20070134796, incorporated herein in its entirety. Additional restriction enzymes also contain separable binding and cleavage domains, and these are contemplated by the present disclosure. See, for example, Roberts et al. (2003) Nucleic Acids Res. 31:418-420.

In certain embodiments, the cleavage domain comprises one or more engineered cleavage half-domain (also referred to as dimerization domain mutants) that minimize or prevent homodimerization, as described, for example, in U.S. Patent Publication Nos. 20050064474; 20060188987 and 20080131962, the disclosures of all of which are incorporated by reference in their entireties herein.

Alternatively, nucleases may be assembled in vivo at the nucleic acid target site using so-called “split-enzyme” technology (see e.g. U.S. Patent Publication No. 20090068164). Components of such split enzymes may be expressed either on separate expression constructs, or can be linked in one open reading frame where the individual components are separated, for example, by a self-cleaving 2A peptide or IRES sequence. Components may be individual zinc finger binding domains or domains of a meganuclease nucleic acid binding domain.

### Zinc Finger Nucleases

In specific embodiments, a chimeric polypeptide is a custom-designed zinc finger nuclease (ZFN) that may be designed to deliver a targeted site-specific double-strand DNA break into which an exogenous nucleic acid, or donor DNA, may be integrated (See co-owned US Patent publication 20100257638, incorporated by reference herein). ZFNs are chimeric polypeptides containing a non-specific cleavage domain from a restriction endonuclease (for example, FokI) and a zinc finger DNA-binding domain polypeptide. See, e.g., Huang et al. (1996) J. Protein Chem. 15:481-9; Kim et al. (1997a) Proc. Natl. Acad. Sci. USA 94:3616-20; Kim et al. (1996) Proc. Natl. Acad. Sci. USA 93:1156-60; Kim et al. (1994) Proc Natl. Acad. Sci. USA 91:883-7; Kim et al. (1997b) Proc. Natl. Acad. Sci. USA 94:12875-9; Kim et al. (1997c) Gene 203:43-9; Kim et al. (1998) Biol. Chem. 379:489-95; Nahon and Raveh (1998) Nucleic Acids Res. 26:1233-9; Smith et al. (1999) Nucleic Acids Res. 27:674-81. In some embodiments, the ZFNs comprise non-canonical zinc finger DNA binding domains (see co-owned US Patent publication 20080182332, incorporated by reference herein). The FokI restriction endonuclease must dimerize via the nuclease domain in order to cleave DNA and introduce a double-strand break. Consequently, ZFNs containing a nuclease domain from such an endonuclease also require dimerization of the nuclease domain in order to cleave target DNA. Mani et al. (2005) Biochem. Biophys. Res. Commun. 334:1191-7; Smith et al. (2000) Nucleic Acids Res. 28:3361-9. Dimerization of the ZFN can be facilitated by two adjacent, oppositely oriented DNA-binding sites. Id.

In particular examples, a method for the site-specific integration of an exogenous nucleic acid into at least one GOI (e.g., herbicide-resistance genes, ALS gene) of a host comprises introducing into a cell of the host a ZFN, wherein the ZFN recognizes and binds to a target nucleotide sequence, wherein the target nucleotide sequence is comprised within at least one GOI of the host. In certain examples, the target nucleotide sequence is not comprised within the genome of the host at any other position than the at least one GOI. For example, a DNA-binding polypeptide of the ZFN may be engineered to recognize and bind to a target nucleotide sequence identified within the at least one GOI (e.g., by sequencing the GOI). A method for the site-specific integration of an exogenous nucleic acid into at least one GOI performance locus of a host that comprises introducing into a cell of the host a ZFN may also comprise introducing into the cell an exogenous nucleic acid, wherein recombination of the exogenous nucleic acid into a nucleic acid of the host comprising the at least one GOI is facilitated by site-specific recognition and binding of the ZFN to the target sequence (and subsequent cleavage of the nucleic acid comprising the GOI).

### Heterologous Nucleic Acid Molecules for Site-Specific Integration

As noted above, insertion of an exogenous sequence (also called a “donor sequence” or “donor” or “transgene”) is provided, for example for expression of a polypeptide, correction of a mutant gene or for increased expression of a wild-type gene. It will be readily apparent that the donor sequence is typically not identical to the genomic sequence where it is placed. A donor sequence can contain a non-homologous sequence flanked by two regions of homology to allow for efficient HDR at the location of interest. Additionally, donor sequences can comprise a vector molecule containing sequences that are not homologous to the region of interest in cellular chromatin. A donor molecule can contain several, discontinuous regions of homology to cellular chromatin. For example, for targeted insertion of sequences not normally present in a region of interest, said sequences can be present in a donor nucleic acid molecule and flanked by regions of homology to sequence in the region of interest. In an exemplary embodiment resistance to ALS-inhibiting herbicides in dandelions is generated by introducing mutations into the ALS protein including, for example, at positon Ala122, Pro197, Ala205, Trp574, Ser653, Asp376, Arg377, Gly654 and combinations thereof.

The donor polynucleotide can be DNA or RNA, single-stranded or double-stranded and can be introduced into a cell in linear or circular form. See e.g., U.S. Patent Publication Nos. 20100047805, 20110281361, 20110207221 and U.S. application Ser. No. 13/889,162. If introduced in linear form, the ends of the donor sequence can be protected (e.g. from exonucleolytic degradation) by methods known to those of skill in the art. For example, one or more dideoxynucleotide residues are added to the 3′ terminus of a linear molecule and/or self-complementary oligonucleotides are ligated to one or both ends. See, for example, Chang et al. (1987) Proc. Natl. Acad. Sci. USA 84:4959-4963; Nehls et al. (1996) Science 272:886-889. Additional methods for protecting exogenous polynucleotides from degradation include, but are not limited to, addition of terminal amino group(s) and the use of modified internucleotide linkages such as, for example, phosphorothioates, phosphoramidates, and O-methyl ribose or deoxyribose residues.

A polynucleotide can be introduced into a cell as part of a vector molecule having additional sequences such as, for example, replication origins, promoters and genes encoding antibiotic resistance. Moreover, donor polynucleotides can be introduced as naked nucleic acid, as nucleic acid complexed with an agent such as a liposome or poloxamer, or can be delivered by viruses (e.g., adenovirus, AAV, herpesvirus, retrovirus, lentivirus and integrase defective lentivirus (IDLV)).

The donor is generally integrated so that its expression is driven by the endogenous promoter at the integration site, namely the promoter that drives expression of the endogenous gene into which the donor is integrated (e.g., herbicide resistance genes). However, it will be apparent that the donor may comprise a promoter and/or enhancer, for example a constitutive promoter or an inducible or tissue specific promoter.

Furthermore, although not required for expression, exogenous sequences may also include transcriptional or translational regulatory sequences, for example, promoters, enhancers, insulators, internal ribosome entry sites, sequences encoding 2A peptides and/or polyadenylation signals.

### Nucleic Acid Molecules Comprising a Nucleotide Sequence Encoding a Targeting Endonuclease

In some embodiments, a nucleotide sequence encoding a targeting endonuclease may be engineered by manipulation (e.g., ligation) of native nucleotide sequences encoding polypeptides comprised within the targeting endonuclease. For example, the nucleotide sequence of a gene encoding a protein comprising a DNA-binding polypeptide may be inspected to identify the nucleotide sequence of the gene that corresponds to the DNA-binding polypeptide, and that nucleotide sequence may be used as an element of a nucleotide sequence encoding a targeting endonuclease comprising the DNA-binding polypeptide. Alternatively, the amino acid sequence of a targeting endonuclease may be used to deduce a nucleotide sequence encoding the targeting endonuclease, for example, according to the degeneracy of the genetic code.

In exemplary nucleic acid molecules comprising a nucleotide sequence encoding a targeting endonuclease, the last codon of a first polynucleotide sequence encoding a nuclease polypeptide, and the first codon of a second polynucleotide sequence encoding a DNA-binding polypeptide, may be separated by any number of nucleotide triplets, e.g., without coding for an intron or a “STOP.” Likewise, the last codon of a nucleotide sequence encoding a first polynucleotide sequence encoding a DNA-binding polypeptide, and the first codon of a second polynucleotide sequence encoding a nuclease polypeptide, may be separated by any number of nucleotide triplets. In these and further embodiments, the last codon of the last (i.e., most 3′ in the nucleic acid sequence) of a first polynucleotide sequence encoding a nuclease polypeptide, and a second polynucleotide sequence encoding a DNA-binding polypeptide, may be fused in phase-register with the first codon of a further polynucleotide coding sequence directly contiguous thereto, or separated therefrom by no more than a short peptide sequence, such as that encoded by a synthetic nucleotide linker (e.g., a nucleotide linker that may have been used to achieve the fusion). Examples of such further polynucleotide sequences include, for example and without limitation, tags, targeting peptides, and enzymatic cleavage sites. Likewise, the first codon of the most 5′ (in the nucleic acid sequence) of the first and second polynucleotide sequences may be fused in phase-register with the last codon of a further polynucleotide coding sequence directly contiguous thereto, or separated therefrom by no more than a short peptide sequence.

A sequence separating polynucleotide sequences encoding functional polypeptides in a targeting endonuclease (e.g., a DNA-binding polypeptide and a nuclease polypeptide) may, for example, consist of any sequence, such that the amino acid sequence encoded is not likely to significantly alter the translation of the targeting endonuclease. Due to the autonomous nature of known nuclease polypeptides and known DNA-binding polypeptides, intervening sequences will not in examples interfere with the respective functions of these structures.

### Use in Breeding Methods

Applicants have surprising discovered that the rubber producing TKS species of are incompatible and do NOT cross with the traditional dandelion weed Taraxacum officinale. Thus, the possibility of herbicide resistant rubber producing dandelion species will not have an deleterious effects on those who wish to use herbicide to kill the common weed.

The transformed plants of the invention may be used in a plant breeding program. The goal of plant breeding is to combine, in a single variety or hybrid, various desirable traits. For field crops, these traits may include, for example, resistance to diseases and insects, tolerance to heat and drought, reduced time to crop maturity, greater yield, and better agronomic quality. With mechanical harvesting of many crops, uniformity of plant characteristics such as germination and stand establishment, growth rate, maturity, and plant height is desirable. Traditional plant breeding is an important tool in developing new and improved commercial crops. This invention encompasses methods for producing a plant by crossing a first parent plant with a second parent plant wherein one or both of the parent plants is a transformed plant according to the invention displaying Fusarium resistance as described herein.

Plant breeding techniques known in the art and used in a plant breeding program include, but are not limited to, recurrent selection, bulk selection, mass selection, backcrossing, pedigree breeding, open pollination breeding, restriction fragment length polymorphism enhanced selection, genetic marker enhanced selection, doubled haploids, and transformation. Often combinations of these techniques are used.

The development of hybrids in a plant breeding program requires, in general, the development of homozygous inbred lines, the crossing of these lines, and the evaluation of the crosses. There are many analytical methods available to evaluate the result of a cross. The oldest and most traditional method of analysis is the observation of phenotypic traits. Alternatively, the genotype of a plant can be examined.

A genetic trait which has been engineered into a particular plant using transformation techniques can be moved into another line using traditional breeding techniques that are well known in the plant breeding arts. For example, a backcrossing approach is commonly used to move a transgene from a transformed maize plant to an elite inbred line, and the resulting progeny would then comprise the transgene(s). Also, if an inbred line was used for the transformation, then the transgenic plants could be crossed to a different inbred in order to produce a transgenic hybrid plant. As used herein, “crossing” can refer to a simple X by Y cross, or the process of backcrossing, depending on the context.

The development of a hybrid in a plant breeding program involves three steps: (1) the selection of plants from various germplasm pools for initial breeding crosses; (2) the selfing of the selected plants from the breeding crosses for several generations to produce a series of inbred lines, which, while different from each other, breed true and are highly uniform; and (3) crossing the selected inbred lines with different inbred lines to produce the hybrids. During the inbreeding process, the vigor of the lines decreases. Vigor is restored when two different inbred lines are crossed to produce the hybrid. An important consequence of the homozygosity and homogeneity of the inbred lines is that the hybrid created by crossing a defined pair of inbreds will always be the same. Once the inbreds that give a superior hybrid have been identified, the hybrid seed can be reproduced indefinitely as long as the homogeneity of the inbred parents is maintained. Transgenic plants of the present invention may be used to produce, e.g., a single cross hybrid, a three-way hybrid or a double cross hybrid. A single cross hybrid is produced when two inbred lines are crossed to produce the F1 progeny. A double cross hybrid is produced from four inbred lines crossed in pairs (A×B and C×D) and then the two F1 hybrids are crossed again (A×B)×(C×D). A three-way cross hybrid is produced from three inbred lines where two of the inbred lines are crossed (A×B) and then the resulting F1 hybrid is crossed with the third inbred (A×B)×C. Much of the hybrid vigor and uniformity exhibited by F1 hybrids is lost in the next generation (F2). Consequently, seed produced by hybrids is consumed rather than planted

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims. Thus, many modifications and other embodiments of the invention will come to mind to one skilled in the art to which this invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims.

### EXAMPLES

### Example 1 —Agrobacterium Rhizogenes-Mediated Transformation in Rubber Producing Dandelions

Several studies have focused on the tissue culture and transformation of Taraxacum, and plants of various Taraxacum species have been regenerated from several tissue types, roots being the most favorable explants with the highest regeneration efficiency (Bowes, 1970, 1976; Lee et al., 2004; Bae et al., 2005). The high regeneration ability of roots is consistent with the well characterized ability of dandelion to vegetatively propagate from root fragments under natural conditions. However, the micro propagation of dandelions still reported different hormone treatments at multiple regeneration stages. Two approaches have been used in dandelion transformation. Agrobacterium tumefaciens has been used to transform Taraxacum mongoliam, Taraxacum platycarpum and TB (Song et al., 1991; Bae et al., 2005; Post et al., 2012), while Agrobacterium rhizogenes has been used to transform T. platycarpum (Lee et al., 2004). A. rhizogenes differs from A. tumefaciens in that it contains native bacterial rol genes, which are often co-transformed with genes of interest. These genes alter endogenous plant hormone concentrations, promoting rapid root growth and increasing the rate of regeneration (Pavli and Skaracis, 2010). We expect the acceleration of root growth to be most prevalent in transformed tissues, allowing transformed cells to persist and better compete for resources, resulting in a more rapid transformation system. Changes in root morphology and biomass followed by A. rhizogenes-mediated transformation had been observed in transgenic T. platycarpum (Lee et al., 2004). To date, while TK and TB have been transformed using leaf tissue as the explant, multiple steps, including callus induction, shoot elongation and root induction, were required during the regeneration stage (Post et al., 2012; Collins-Silva et al., 2012). Applicants demonstrate herein, the strong regeneration capacity of dandelion roots under tissue culture conditions without the addition of plant hormones was used to generate previously undescribed protocols for A. rhizogenes-mediated transformation in Taraxacum. Using these methods, genes encoding green fluorescent protein (GFP) and cyan fluorescent protein (CFP) were transformed into TK and TB to yield non-composite transgenic lines in a short period of time. The methods described here offer a highly efficient and fast approach to generate transgenic plants without hormone treatments and without a callus stage.

### Materials and Methods

### Plant Materials

Seeds of TK from USDA accession KAZ08-017 (W6 35172) and an apomictic TB lineage donated by Peter van Dijk (Keygene, Wageningen, Netherlands), designated as Clone A, were used (Kirschner et al., 2013). Seeds were surface-sterilized with 70% ethanol for 2 min, followed by soaking in a 0.25% sodium hypochlorite solution with 0.5% sodium dodecyl sulfate for 10 min. Seeds then were rinsed with autoclaved water 5 times and germinated on solid half strength Murashige and Skoog (½ MS) medium (½ strength MS micro- and macro-salts (Caisson Laboratories, Inc., North Logan, Utah, USA) supplemented with full strength Gamborg's B5 vitamins, 10 g L-1 sucrose and 8 g L-1 agar (Sigma-Aldrich®, St. Louis, Mo., USA)) (Murashige and Skoog, 1962; Gamborg et al., 1968). The plants were maintained at 23-27° C. under 16 h light/8 h dark photoperiod with a light intensity of 30 μmol m⁻² s⁻¹ using white-fluorescent tubes and grown for 12 weeks.

### Binary Vector and Agrobacterium Strain

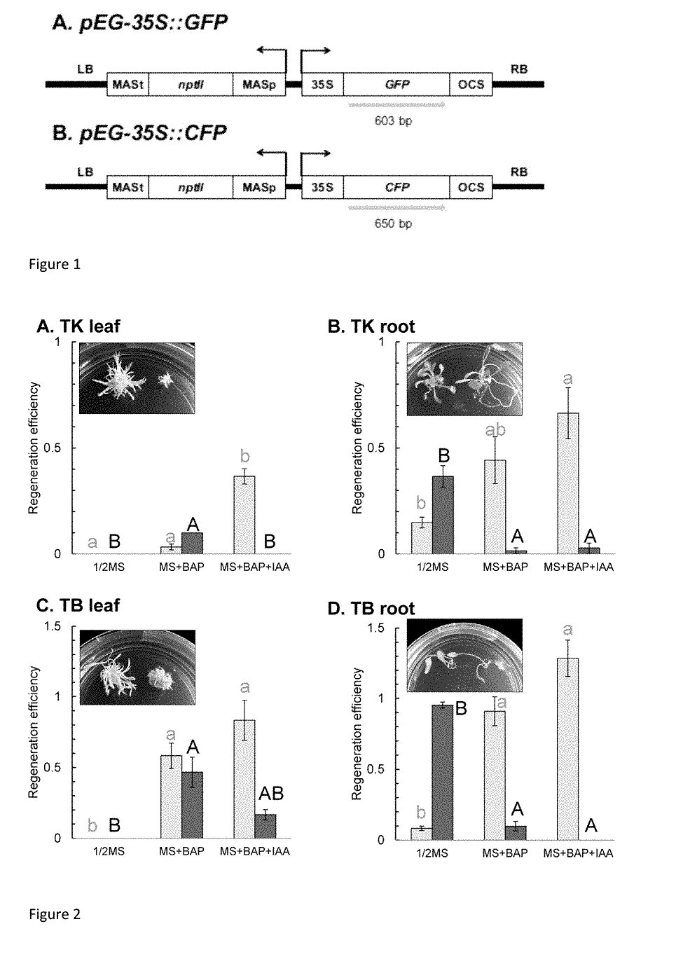

The pEarleyGate 100 series vector (Arabidopsis Biological Resource Center (ABRC) stock number: CD3-724) was amended by replacing the glufosinate resistance gene with the kanamycin resistance gene neomycin phosphotransferase II (nptII) as the selective marker (Earley et al., 2006). Kanamycin was used instead of glufosinate, as it is considered more ecologically innocuous and is more commonly used for selection in dicotyledonous plants (Nap et al., 1992; Miki and McHugh, 2004). Genes encoding GFP and CFP were amplified using high fidelity Platinum® Taq DNA Polymerase (Invitrogen™, Carlsbad, Calif., USA) from pEarleyGate vectors sourced from Ohio State's Arabidopsis Biological Resource Center (ABRC stock number CD3-685 and CD3-684, respectively). Amplicons then were cloned into the modified pEarleyGate 100 vector, using the PCR8/GW/TOPO Cloning Kit and LR Clonase (Invitrogen™, Carlsbad, Calif., USA) according to manufacturer's instructions, termed as pEG-35S::GFP (FIG. 1A) and pEG-35S::CFP (FIG. 1B). Expression vectors were introduced into A. rhizogenes K599 wild-type (kindly provided by Prof. John Finer, The Ohio State University, OARDC, Wooster, Ohio, USA) by electroporation. A. rhizogenes, harboring expression constructs, was grown for 36 h in liquid YEP medium (10 g L-1 yeast extract, 10 g L-1 peptone, 5 g L-1 NaCl), containing 100 mg L-1 kanamycin, shaken at 150 rpm at 28° C. The Agrobacteria cultures were then pelleted and washed sequentially with liquid YEP medium and ½ MS medium containing 200 μM acetosyringone. Agrobacteria cultures finally were suspended in liquid ½ MS medium containing 200 μM acetosyringone with OD₆₀₀ 0.6 for transformation.

### Optimization of Explants and Regeneration System

Different explants and regeneration media were used to optimize regeneration efficiency. Untransformed 1-2 cm root fragments and 1 cm2 leaf discs of TK USDA line 17 and TB were grown on three different regeneration media, ½ strength MS medium (½ MS), full-strength MS medium (MS, full strength MS micro- and macro-salts with Gamborg's B5 vitamins, 20 g L-1 sucrose and 8 g L-1 agar) supplemented with 1 mg L-1 6-benzylaminopurine (BAP) (MS+BAP), MS medium supplemented with 1 mg L-1 BAP and 0.2 mg L-1 indole-3-acetic acid (IAA) (MS+BAP+IAA). The above BAP concentration in MS+BAP medium was selected as it has previously been reported to give the highest shoot formation efficiency from non-transformed and A. rhizogenes transformed T. platycarpum roots (Lee et al., 2004). Additionally, hormone concentrations in MS+BAP+IAA medium were selected based on reported shooting medium used for TK shoot regeneration (Collins-Silva et al., 2012). Approximately, 50 root fragments and 20 leaf discs were used for each replicate and three replicates were set for each medium. After 30 days regeneration, regenerated calli and shoot numbers were recorded to calculate regeneration efficiency.

### Inoculation, Co-Culture and Selection

Root fragments of TK and TB were cut from 12-week-old plants and inoculated with A. rhizogenes harboring GFP and CFP expression vectors by mixing on a shaker at 100 rpm for 15 min. Roots then were blotted dry on filter paper and transferred to co-culture medium (solid ½ MS medium with 200 μm acetosyringone). After 3 days of co-culture with agrobacteria, root fragments were washed sequentially with water and liquid ½ MS medium with 400 mg L⁻¹ Timentin, and then transferred to solid ½ MS medium with 400 mg L⁻¹ Timentin. After 1 week of recovery, TK root fragments were washed with liquid ½ MS medium with 400 mg L⁻¹ Timentin and 5 mg L⁻¹ kanamycin and then transferred to plates with ½ MS medium with 400 mg L⁻¹ Timentin and 5 mg L⁻¹ kanamycin. After 1 week of recovery, TB root fragments were washed with liquid ½ MS medium with 400 mg L⁻¹ Timentin and 15 mg L⁻¹ kanamycin and then transferred to plates with ½ MS medium with 400 mg L⁻¹ Timentin and 15 mg L⁻¹ kanamycin. Roots were separated into two groups by diameter (D<1 mm and D≧1 mm) and grown on ½ MS medium with selection for about 4 weeks. Regenerated plantlets with hairy root phenotypes were transferred to solid ½ MS medium with 400 mg L⁻¹ Timentin and 10 mg L⁻¹ kanamycin for TK while 20 mg L⁻¹ kanamycin was used for TB. After 3 weeks further selection, transgene events were validated in selected plants. Root fragments of TK and TB were also inoculated with A. rhizogenes K599 wild type using the same method. After recovery, root fragments were transferred to ½ MS medium with 400 mg L⁻¹ Timentin for regeneration. Approximately 30 root fragments were used for each replicate and three replicates were used for each treatment.

### PCR and Reverse Transcription PCR

Putative transgenic plants were validated by polymerase chain reaction (PCR) of GFP or CFP. Total genomic DNA was extracted from leaves of plants transformed with K599 harboring fluorescent protein expression vectors as well as leaves of non-transgenic plants as negative controls. A 2% CTAB method was scaled to a 96 well format using the GenoGrinder platform (SPEX, Metuchen, N.J., USA) for DNA extraction (Kabelka et al., 2002). PCR was performed in a 15 μL reaction containing 1× Standard Taq Reaction Buffer, 200 μM dNTPs, 0.2 μM forward and 0.2 μM reverse primers, 0.4 U Taq DNA Polymerase and 10 ng DNA. Primers used to amplify 603 bp region of GFP were vGFP forward: 5′-AGAGGGTGAAGGTGATGCAA-3′ (SEQ ID NO:1) and vGFP reverse: 5′-CCATGTGTAATCCCAGCAGC-3′ (SEQ ID NO:2); the 650 bp region of CFP was amplified using primers vCFP forward: 5′-TAAACGGCCACAAGTTCAGC-3′ (SEQ ID NO:3) and vCFP reverse: 5′-CTTGTACAGCTCGTCCATGC-3′ (SEQ ID NO:4). PCR procedures used were 5 min initial denaturation at 95° C., 30 s denaturation at 95° C., 30 s annealing at 54° C., 60 s elongation at 68° C. for 35 cycles, followed by final extension at 68° C. for 5 min. A total volume of 10 μL PCR products was loaded on 2% agarose gels (w/v) with ethidium bromide for electrophoresis. All the reagents were obtained from New England Biolabs Inc., Ipswich, Mass., USA.

Total RNA was extracted from leaves of plants transformed with K599 harboring fluorescent protein expression vectors, as well as leaves of non-transgenic plants as negative controls, following the method described by Chomczynski and Sacchi (2006). RNA from each sample were treated by DNase I using TURBO DNA-Free™ Kit to Remove DNA (Invitrogen™, Carlsbad, Calif., USA). First-strand cDNA was synthesized using SuperScript™ II Reverse Transcriptase (Invitrogen™, Carlsbad, Calif., USA). The amount of 50 ng cDNA was used for reverse transcription PCR (RT-PCR) using reactions and procedures described above for GFP and CFP transformants, as well as the following primers: RT-GFP forward: 5′-AGAGGGTGAAGGTGATGCAA-3′ (SEQ ID NO:5); RT-GFP reverse: 5′-CTCTTGAAGAAGTCGTGCCG-3′ (SEQ ID NO:6); RT-CFP forward 5′-CACATGAAGCAGCACGACTT-3′ (SEQ ID NO:7); RT-CFP reverse 5′-TCCTTGAAGTCGATGCCCTT-3′ (SEQ ID NO:8). Endogenous gene □-actin (ACTB) was amplified using the same amount of cDNA and primers: ACTB forward: 5′-AGCAACTGGGATGACATGGA-3′ (SEQ ID NO:9); ACTB reverse: 5′-CATACATGGCGGGGACATTG-3′ (SEQ ID NO:10). A total volume of 10 μL PCR products were loaded on 2% agarose gels (w/v) with ethidium bromide for electrophoresis. All the reagents which were not specifically mentioned above were obtained from New England Biolabs Inc., Ipswich, Mass., USA.

### Fluorescent Protein Visualization

Fluorescent protein functional expression was confirmed for both leaf and root tissue using a confocal scanning microscope (Molecular and Cellular Imaging Center, The Ohio State University, OARDC, Wooster, Ohio, USA). After 8 weeks of selection, root and leaf samples from non-transgenic plants, as well as from PCR and RT-PCR confirmed transgenic plants, were placed in glass bottom dishes. Samples were covered with glass cover slips and water was added between the bottom of dishes and the glass cover. Samples were placed under a Leica TCS SP5 confocal scanning microscope and images were captured using Leica Application Suite Advanced Florescent software. GFP images were captured under excitation laser Argon-blue (488 nm and 514 nm) with excitation wavelengths 488 nm at 82% laser intensity. Images were collected from 497 nm to 557 nm with 865 smart gain and 50.1 μm pinhole. CFP were visualized under UV (405 nm) laser with 77% laser intensity. Images were collected from 453 nm to 531 nm with 845 smart gain and 64.9 μm pinhole. Figures were created by Microsoft PowerPoint (version 14.0.7128.5000).

### Subculture of Validated Plants and Analysis of Transgene Inheritance

Hairy roots that were greater than 1 cm long, with a diameter greater than 1 mm, from transformed plants validated by PCR, RT-PCR and microscopy, were placed on ½ MS medium with 400 mg L-1 Timentin and 10 mg L-1 kanamycin for TK and 20 mg L-1 kanamycin for TB. At least 2 new plantlets were generated for each event before transitioning the transgenic event to non-sterile conditions.

Validated transformed plants were transferred into sterile peat pellets soaked with liquid ½ MS medium with 400 mg L-1 Timentin. After two weeks, the media in the peat pellets was replaced with water and the transgenic plants with peat pellets were transferred to micro propagation trays, where the humidity was lowered over a period of 1 week. Transformed plants in peat pellets then were transferred into 3.8 L pots filled with Pro-mix and then moved into a growth chamber with a 12 h light/12 h dark photoperiod, light intensity of 400 μmol m-2 s-1, at 22° C., and relative humidity of 80%. After 1 month, transgenic TK plants were reciprocally crossed with at least three different genotypes of non-transgenic TK to obtain T1 populations. Seeds were collected 15 days after pollination. These seeds were germinated in Pro-mix and leaves were collected 20 days after germination for DNA extraction. DNA was extracted and CFP amplified using the methods described previously.

### Statistical Analysis

Regeneration efficiency was calculated using the number of regenerated shoots, calli or plants over the number of starting leaf discs or root fragments. Treatment effects were detected using one-way analysis of variance (ANOVA) and Tukey's HSD multiple comparison of mean test by R (R Core Team, 2013). Influence of root size on regeneration efficiency was analyzed using vectors as a random factor. Significant differences were claimed at P<0.05.

FIG. 1: Binary vectors for green fluorescent protein

### Results and Discussion

### Selection of Regeneration Media and Explants

To investigate the optimal medium and explant for TK and TB to achieve highest regeneration efficiency, three different media treatments were used to determine their ability to mediate the regeneration plants from leaf discs and root fragments. These three media had different effects on both TK and TB regeneration efficiency from leaf discs. The MS+BAP and MS+BAP+IAA media induced calli from leaf edges whereas ½ MS medium did not induce calli, shoots, or roots from leaf discs. When using roots as explants, MS+BAP and MS+BAP+IAA induced callus production as well, with few shoots appearing on calli. Fragments regenerated on ½ MS medium with no addition of hormones were able to generate plantlets in a period of 14 days (FIG. 2 A-D). Direct shooting from explants was considered an ideal approach for plant regeneration, as it both shortens the regeneration cycle and limits the introduction of undesired somaclonal variation, which can occur in the callus phase (Nwauzoma and Jaja, 2013). Plantlets regenerated from root fragments on ½ MS medium were able to develop more quickly and vigorously than other methods (FIG. 2 A-D). While shoots were induced on other media using either roots or leaf discs, under these conditions a short callus stage was observed prior to the appearance of shoots. Additionally, these shoots were much smaller than shoots induced from roots on ½ MS medium and were not able to develop to plantlets without using rooting media. Therefore, the use of root tissues and ½ MS medium were selected as optimal recovery conditions for downstream generation of transformed plants. While the hormone-free transformation method here provides several advantages, it must be noted that our studies only incorporated phytohormones (IAA and BAP) at the concentrations described above. Since, callus tissues do exhibit sensitivity to gradients in hormone concentrations, it is entirely possible that the inclusion of either IAA or BAP at lower or higher concentrations might provide additional advantages in the regeneration of transgenic tissues (i.e., increased growth or the production of additional root mass) which would not have been revealed in our assays. As the hormone-free regeneration method provided a rapid and simple approach for TK and TB regeneration, however, this method was selected as a focus for the current study. One advantage to this method is that it circumvents iterative hormone treatments requiring transfer to several different media accompanied by manual manipulations. Additionally, this method also maintained a high regeneration efficiency by reducing the number of steps required and the potential losses and costs associated with them.

FIG. 2: Effects of different explants (leaf disc and root) and three media (½ MS, MS+BAP and MS+BAP+IAA) on Taraxacum kok-saghyz (TK) and T brevicorniculatum (TB) regeneration efficiency.

### Regeneration Capability of Transgenic and Non-Transgenic Roots

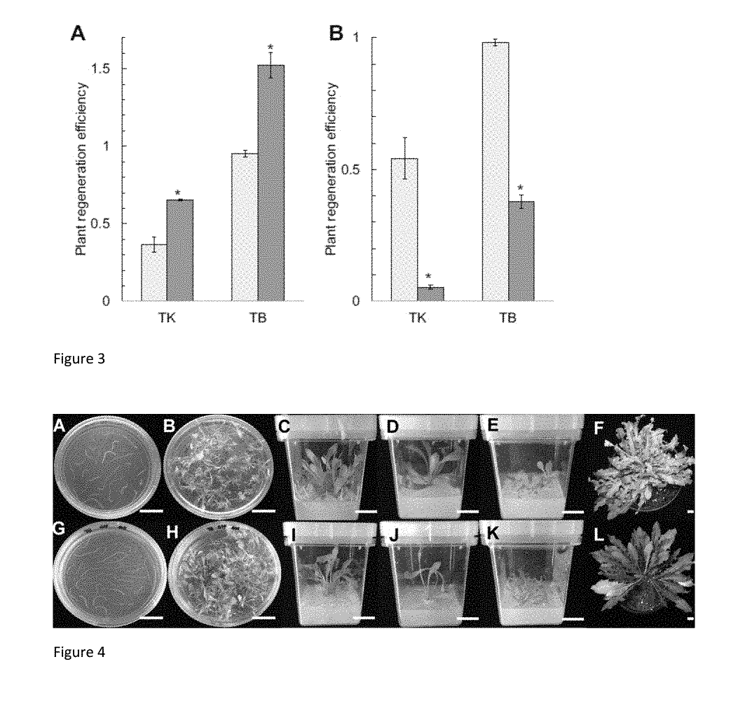

Plant root fragments were first transformed using A. rhizogenes wild type strain K599. Shoot emergence was observed 10 days after transformation, followed by the formation of hairy roots. Within one month, TK plantlets were obtained with 65.3±0.7% regeneration efficiency, which was 28.7% higher than the regeneration efficiency seen in non-inoculated plants (36.6±5.1%) (FIG. 3A). TB regeneration efficiency reached 152.3±8.2% (more than one shoot emerged from a single root fragment) with inoculation, significantly higher than the regeneration efficiency of 95.2±2.2% without inoculation (FIG. 3A), a phenomenon we suspect is due primarily to both the strong regenerative ability of TB and the rapid growth and differentiation induced by hairy root transformation.

FIG. 3: Effects of inoculation and root size on Taraxacum kok-saghyz (TK) and T. brevicorniculatum (TB) regeneration efficiency.

### Selection and Regeneration of GFP and CFP Transgenic Plants

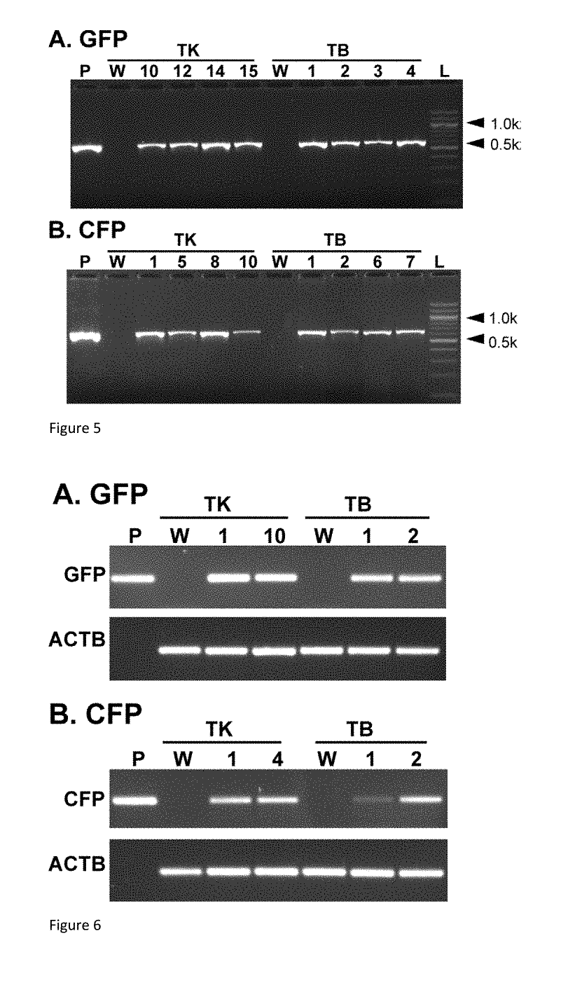

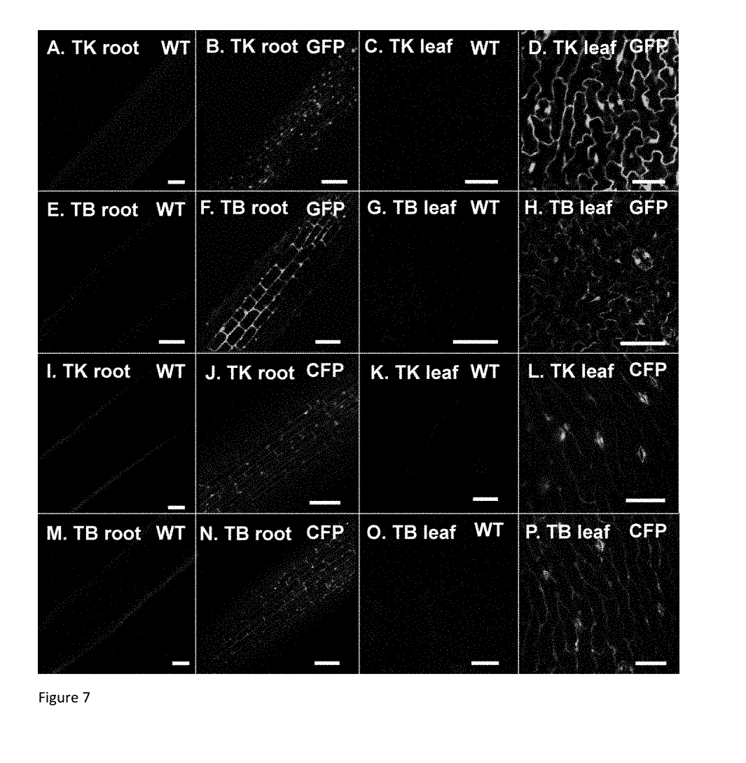

To achieve efficient selection for transgenic plantlets, we tested a range of concentrations and identified 10 mg L⁻¹ and 20 mg L⁻¹ as kanamycin concentrations effective at eliminating non-transgenic TK or TB, respectively (data not shown). Plants able to survive under 10 mg L⁻¹ (TK) or 20 mg L⁻¹ (TB) kanamycin could be obtained within 8 weeks after selection, (FIGS. 4A,-C and G-I). Compared to non-transgenic plants (FIGS. 4D and J), transformed plants exhibited hairy root phenotypes, including wrinkled and high density leaves as well as plagiotropic and extensively branched roots. Transgene presence was validated by PCR analysis (FIGS. 5A and B) and transgene expression at the transcription level was confirmed by RT-PCR (FIGS. 6A and B) using leaf tissue. At the tissue level, confocal microscopy of transgenic root and leaf tissues showed transformation of both tissue types (FIG. 7A-P). GFP and CFP were shown to express stably in the protoplasm and nuclei of root and leaf tissues. It is important to note, however, that due to slight differences in organ morphology between TK and TB, the fluorescence intensity in the images is not quantitative; i.e., the increased fluorescence intensity observed in TB vs. TK roots may not indicate higher GFP or CFP expression. Collectively, transgenes were present and functionally expressed in both leaf and root tissue, suggesting that the kanamycin concentrations used for selection were sufficient to produce non-composite plants. However, composite plants may be useful in basic research to evaluate transport phenomena between roots and shoots (Ko et al., 2014). Moreover, large scale production of secondary metabolites could be achieved using hairy roots from composite plants, particularly for lethal transgene events or species with poor regeneration ability (Benabdoun et al., 2011).

### Influence of Root Size on Regeneration Efficiency and Transformation Efficiency

To investigate the influence of root size on regeneration efficiency and transformation efficiency, two size categories of root fragments were used for GFP and CFP transformation. We found that root diameter significantly impacted (P<0.05) the recovery of transformants. In TK, young adventitious roots with diameters <1 mm were generally unable to regenerate plantlets, while more mature roots ≧1 mm exhibited a higher rate of regeneration (FIG. 3B). Interestingly, in contrast to TK, TB roots with diameters <1 mm showed strong regenerative ability, although this was still lower than regeneration observed using larger roots (FIG. 3B). We have observed that larger root systems can be obtained by adding hormones such as indole-3-butyric acid to growth media. We expect that root fragments taken from such plants would have similarly favorable regenerative abilities. On average, transformation efficiency (number of transgenic plants/number of root fragments) of roots with diameters ≧1 mm was 24.7% and 15.7% for TK and TB, respectively; about seven independent transgenic events were generated per starting plant for TK and four for TB.

The TK germplasm selected for this research, USDA accession KAZ08-017 (W6 35172), exhibited average regeneration abilities from both shoots and roots (data not shown) comparable to those observed in other KAZ accessions. While the transformation of other TK accessions was not tested in this research, given the average regeneration rate of KAZ08-017, the methods described here are likely to be successful when applied to other TK accessions.

### Subculture, Acclamation and Inheritance Analysis of Validated Transgenic Plants

Taraxacum plants were initially subcultured from leaves, which required multiple steps over a 12 week period (data not shown). An alternative, simpler subculture method from roots was developed. Hairy roots induced by A. rhizogenes infection were excised and moved to ½ MS medium without hormones. After 30 days, plantlets had regenerated and showed hairy root phenotypes, suggesting that the strong regenerative capability of roots was able to tolerate hormonal imbalances potentially introduced by rol genes of A. rhizogenes (FIGS. 4E and 4K). As hairy root transformants generally produce many hairy roots, this system allows for rapid duplication of transgene events.

FIG. 4: The A. rhizogenes-mediated transformation of Taraxacum kok-saghyz (TK) and T. brevicorniculatum (TB) using root fragments as explants.

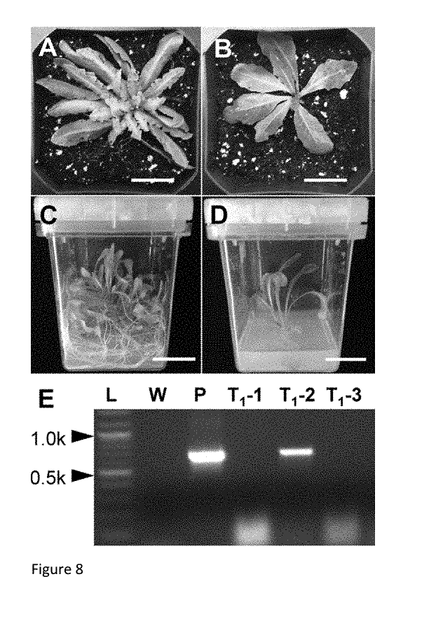

Transgenic plants can be transferred from tissue culture to growth chambers or greenhouses within 21 days. The survival rates of transgenic plants were 95% and 100% for TK and TB, respectively. After recovery and growth in soil for 30 days, transgenic TK plants were able to flower and produce viable progeny in reciprocal crosses (FIGS. 4F and L, FIG. 8A-D). Both hairy root phenotypes and fluorescent protein genes were heritable in the T₁ generation, with segregation (FIG. 8A-E).

The hairy root phenotypes observed in transformed plants persisted after the transition to non-sterile growth in soil (FIGS. 4E and 4J). This growth habit was reported to increase root to shoot biomass ratio and increase the production of secondary metabolites, including both alkaloids and terpenoids (of particular interest, since increases in terpenoid production could potentially increase rubber yields from TK or TB) in several species (Cai et al., 1995; Kim et al., 2002; Srivastava and Srivastava, 2007). While the generation of numerous adventitious roots, instead of a few tap roots, may allow for better competitiveness and utilization of soil nutrients, it also could result in roots that are too fragile to be harvested. Additionally, while the hairy root phenotypes generally increase without secondary metabolism, they may have the potential to affect rubber production or rubber molecular weight. If this growth habit proves to be undesirable, genes of interest can be segregated from native A. rhizogenes events in the T₁ generation. As the integration of native A. rhizogenes genes is independent of the integration of genes of interest, they will generally be inserted in different regions of the genome and will not be linked to each other. Alternatively, A. tumefaciens-mediated transformation method may be achieved using the high efficiency regeneration system described here. The potential implications of a hairy root growth habit and metabolic modification of TK and TB will be evaluated in future work.

### CONCLUSIONS

Applicants present here the development of a novel plant transformation system using A. rhizogenes to transform root tissue efficiently and leveraging the ability of Taraxacum species to regenerate entire plants from root fragments to create a rapid pipeline for the generation of transgenic dandelion lines. The regeneration of plants from root fragments in tissue culture without hormone treatment has not previously been reported in Taraxacum. The method presented here could be used to increase accessibility, reproducibility, and throughput in transformation efforts. Progeny of crosses between TB and TK segregate TK phenotypes, suggesting that transgene events could be moved between species and that TB can serve as a clonal courier of transgene events, where its vigorous growth rate and polyploidy could facilitate challenging transformations. Collectively, these results provide a platform for future transgene events in rubber producing dandelion species that may be used to investigate components of rubber biosynthesis and improve rubber yield as well as agronomic traits.

FIG. 5: Polymerase chain reaction (PCR) analysis of green fluorescent protein (GFP) and cyan fluorescent protein (CFP) in transgenic Taraxacum kok-saghyz (TK) and T. brevicorniculatum (TB) plants.

FIG. 6: Reverse transcription polymerase chain reaction (RT-PCR) analysis of green fluorescent protein (GFP) and cyan fluorescent protein (CFP) expression.

FIG. 7: Stable green fluorescent protein (GFP) and cyan fluorescent protein (CFP) expression in transgenic Taraxacum kok-saghyz (TK) and T. brevicorniculatum (TB) under a Leica TCS SP5 Confocal Microscope.

FIG. 8: Stable inheritance and segregation of hairy root phenotypes and fluorescent protein gene in Taraxacum kok-saghyz (TK) T₁ generation.

### REFERENCES

Bae, T. W., Park, H. R., Kwak, Y. S., Lee, H. Y., Ryu, S. B., 2005. Agrobacterium tumefaciens-mediated transformation of a medicinal plant Taraxacum platycarpum. Plant Cell Tiss. Org. Culture 80, 51-57, http://dx.doi.org/10.1007/s11240-004-8807-7.
Benabdoun, F. M., Nambiar-Veetil, M., Imanishi, L., Svistoonoff, S., Ykhlef, N., Gherbi, H., Franche, C., 2011. Composite actinorhizal plants with transgenic roots for the study of symbiotic associations with Frankia. J. Bot. 2011, 8, http://dx.doi.org/10.1155/2011/702947, Article ID 702947.
Bowes, B. G., 1970. Preliminary observations on organogenesis in Taraxacum officinale tissue cultures. Protoplasma 71, 197-202, http://dx.doi.org/10.1007/BF01294312.
Bowes, B. G., 1976. Polar regeneration in excised roots of Taraxacum officinale Weber: a light and electron microscopic study. Ann. Bot. 40, 423-432.
Cai, G., Li, G., Ye, H., Li, G., 1995. Hairy root culture of Artemisia annua L. by Ri plasmid transformation and biosynthesis of artemisinin. Chin. J. Biotechnol. 11, 227-235.
Chomczynski, P., Sacchi, N., 2006. The single-step method of RNA isolation by acid guanidinium thiocyanate-phenol-chloroform extraction: twenty-something years on. Nat. Protoc. 1, 581-585, http://dx.doi.org/10.1038/nprot.2006.83.
Collins-Silva, J., Nural, A. T., Skaggs, A., Scott, D., Hathwaik, U., Woolsey, R., Schegg, K., McMahan, C., Whalen, M., Cornish, K., Shintani, D., 2012. Altered levels of the Taraxacum kok-saghyz (Russian dandelion) small rubber particle protein, TkSRPP3, result in qualitative and quantitative changes in rubber metabolism. Phytochemistry 79, 46-56, http://dx.doi.org/10.1016/j.phytochem.2012.04.015.
Cornish, K., 2001. Biochemistry of natural rubber, a vital raw material, emphasizing biosynthetic rate, molecular weight and compartmentalization, in evolutionarily divergent plant species (1963 to 2000). Nat. Prod. Rep. 18, 182-189, http://dx.doi.org/10.1039/a902191 d.
Earley, K. W., Haag, J. R., Pontes, O., Opper, K., Juehne, T., Song, K., Pikaard, C. S., 2006. Gateway-compatible vectors for plant functional genomics and proteomics. Plant J. 45, 616-629, http://dx.doi.org/10.1111/j.1365-313X.2005.02617.x.
Edathil, T. T., 1986. South American leaf blight—a potential threat to the natural rubber industry in Asia and Africa. Trop. Pest Manage. 32, 296-303, http://dx.doi.org/10.1080/09670878609371083.
Gamborg, O. L., Miller, R. A., Ojima, K., 1968. Nutrient requirements of suspension cultures of soybean root cells. Exp. Cell Res. 50, 151-158, http://dx.doi.org/10.1016/0014-4827(68)90403-5.
Kabelka, E., Franchino, B., Francis, D. M., 2002. Two Loci from Lycopersicon hirsutum LA407 confer resistance to strains of Clavibacter michiganensis subsp. michiganensis. Phytopathology 92, 504-510, http://dx.doi.org/10.1094/PHYTO.2002.92.5.504.
Kirschner, J., SS\check{}tee\check{}pánek, J., CC\check{}ernyy′, T., Heer, P. D., Dijk van, P. J., 2013. Available ex situ germplasm of the potential rubber crop Taraxacum koksaghyz belongs to a poor rubber producer, T. brevicorniculatum (Compositae-Crepidinae). Genet. Resour. Crop Evol. 60, 455-471, hap://dx.doi.org/10.1007/s10722-012-9848-0.
Kim, Y., Wyslouzil, B. E., Weathers, P. J., 2002. Secondary metabolism of hairy root cultures in bioreactors. In Vitro Cell. Dev. Biol. Plant 38, 1-10, http://dx.doi.org/10.1079/IVP2001243.
Ko, D., Kang, J., Kiba, T., Park, J., Kojima, M., Do, J., Kim, K. Y., Kwon, M., Endler, A., Song, W.-Y., Martinoia, E., Sakakibara, H., 2014. Arabidopsis ABCG14 isessential for the root-to-shoot translocation of cytokinin. PNAS 111, 7150-7155, http://dx.doi.org/10.1073/pnas.1321519111.
Lee, M. H., Yoon, E. S., Jeong, J. H., Choi, Y. E., 2004. Agrobacterium rhizogenes-mediated transformation of Taraxacum platycarpum and changes of morphological characters. Plant Cell Rep. 22, 822-827, http://dx.doi.org/10.1007/s00299-004-0763-5.
Lieberei, R., 2007. South American leaf blight of the rubber tree (Hevea spp.): new steps in plant domestication using physiological features and molecular markers. Ann. Bot. 100, 1125-1142, http://dx.doi.org/10.1093/aob/mcm133.
Miki, B., McHugh, S., 2004. Selectable marker genes in transgenic plants: applications, alternatives and biosafety. J. Biotechnol. 107, 193-232, http://dx.doi.org/10.1016/j.jbiotec.2003.10.011.
Mooibroek, H., Cornish, K., 2000. Alternative sources of natural rubber. Appl. Microbiol. Biotechnol. 53, 355-365, http://dx.doi.org/10.1007/s002530051627.
Murashige, T., Skoog, F., 1962. A revised medium for rapid growth and bio assays with tobacco tissue cultures. Physiol. Plant. 15, 473-497, http://dx.doi.org/10.1111/j.1399-3054.1962.tb08052.x.
Nap, J.-P., Bijvoet, J., Stiekema, W. J., 1992. Biosafety of kanamycin-resistant transgenic plants. Transgenic Res. 1, 239-249, http://dx.doi.org/10.1007/BF02525165.
Nwauzoma, A. B., Jaja, E. T., 2013. A review of somaclonal variation in plantain (Musa spp): mechanisms and applications. J. Appl. Biosci. 67, 5252-5260.
Pavli, O. I., Skaracis, G. N., 2010. Fast and efficient genetic transformation of sugar beet by Agrobacterium rhizogenes. Protoc. Exch., http://dx.doi.org/10.1038/nprot.2010.98.
Post, J., van Deenen, N., Fricke, J., Kowalski, N., Wurbs, D., Schaller, H., Eisenreich, W., Huber, C., Twyman, R. M., Prufer, D., et al., 2012. Laticifer-specific cis-prenyltransferase silencing affects the rubber, triterpene, and inulin content of Taraxacum brevicorniculatum. Plant Physiol. 158, 1406-1417, http://dx.doi.org/10.1104/pp. 111.187880.
R Core Team, 2013. R: A language and environment for statistical computing. R Foundation for Statistical Computing, Vienna, Austria. URL http://www.R-project.org/Song, Y. H., Wong, P. K., Chua, N. H., 1991. Tissue culture and genetic transformation of dandelion. Acta Hortic. (ISHS) 289, 261-262.
Srivastava, S., Srivastava, A. K., 2007. Hairy root culture for mass-production of high-value secondary metabolites. Crit. Rev. Biotechnol. 27, 29-43, http://dx.doi.org/10.1080/07388550601173918.
Van Beilen, J. B., Poirier, Y. 2007. Guayule and Russian dandelion as alternative sources of natural rubber. Critical Reviews in Biotechnology 27, 217-231. doi: 10.1080/07388550701775927.
Whaley, W. G., Bowen, J. S. 1947. Russian dandelion (kok-saghyz): an emergency source of natural rubber (US Department of Agriculture).

### Example 2—Generation of Glufosinate-Resistant Transgenic Rubber-Producing Dandelions

Applicants show the successful generation of rubber-producing dandelions species, which display resistance to broad leaf herbicide glufosinate. Using similar methods as described in Example 1, transgenic plants were generated using Agrobacterium tumafaciens-mediated transformation of leaf discs or root fragments, followed by regeneration of transgenic plants, which were then acclimated and transferred to pots within greenhouses. The bar gene, was expressed singly or in combination with other genes of interest in two rubber-producing dandelion species, Taraxacum kok-saghyz and Taracum brevicorniculatum. Table 1 describes the list of gene constructs used for the generation of transgenic herbicide-resistant dandelions.

TABLE 1
Gene constructs used to express the bar gene and other GOI

| Species | TK | TK | TK | TK | TB | TB |
|---|---|---|---|---|---|---|
| Agro Strain | A. tumefacians | A. tumefacians | A. tumefacians | A. tumefacians | A. tumefacians | A. tumefacians |
| | GV3101 | GV3101 | GV3101 | GV3101 | GV3101 | GV3101 |
| Vector | PEG301 | PEG301 | PEG301 | PEG301 | PEG301 | PEG301 |
| Selection Marker | BAR | BAR | BAR | BAR | BAR | BAR |
| Promoter | CaMV35S | CaMV35S | CaMV35S | GmROOT7 | CaMV35S | GmROOT7 |
| Promoter Source | Cauliflower | Cauliflower | Cauliflower | Glycine max | Cauliflower | Glycine max |
| | Mosaic Virus | Mosaic Virus | Mosaic Virus | | Mosaic Virus | |
| Gene of Interest | SST/FFT | SST/FFT | SST/FFT | HMGR | GFP | GFP |
| (GOI) | | | | | | |
| GOI Source | Taraxacum | Taraxacum | Taraxacum | Saccharomyces | Modified GFP from jellyfish | Modified GFP from jellyfish |
| | kok-saghyz | kok-saghyz | kok-saghyz | cerevisiae | Aequorea victoria | Aequorea victoria |
| Transformed Tissue | Leaf disc | Leaf disc | Leaf disc | Leaf disc | Root fragment | Root fragment |
| Gene Presence | CaMV35S | CaMV35S, | BAR | OCS | GFP | GFP |
| Validation by PCR | | OCS | | | | |
| Shoots in petri | 4 | 6 | 0 | 7 | 1 | 4 |
| dishes | | | | | | |
| Rooting in magenta | 2 | 3 | 1 | 5 | 2 | 4 |
| boxes | | | | | | |
| Black pellet | 30 | 5 | 1 | 11 | 11 | 13 |
| acclimation | | | | | | |
| Growth Chamber | 4 | 5 | 0 | 3 | 0 | 0 |
| Total Transgenic | 40 | 19 | 2 | 26 | 14 | 21 |
| Plants | | | | | | |

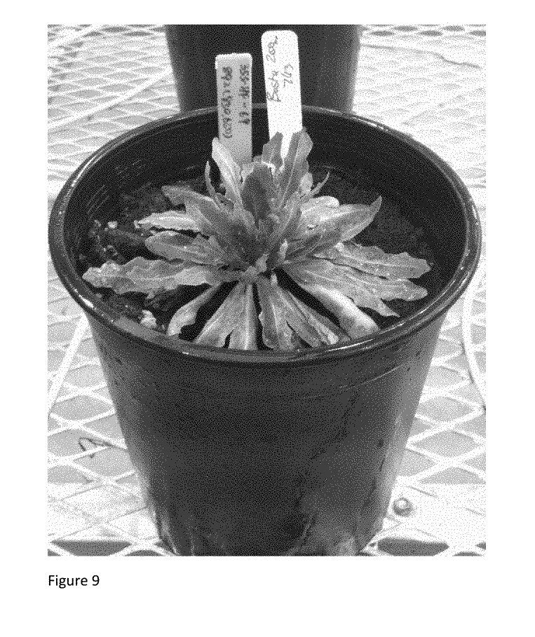

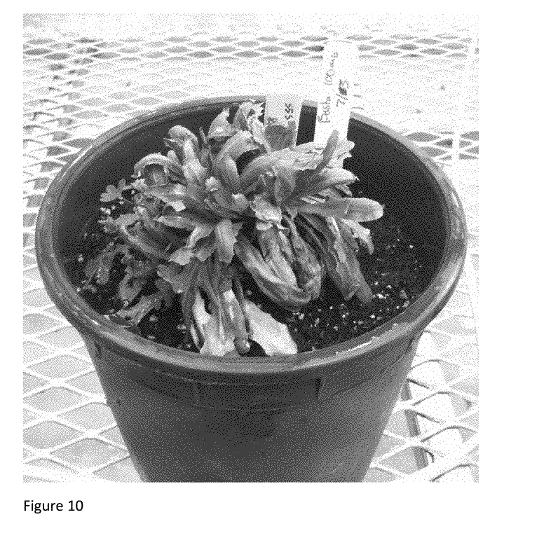

Confirmation of a transgenic event was validated by PCR and resistance confirmed through treatment of mature transgenic plants with varying doses of glufosinate (e.g., 25 mg/L, 50 mg/L, 100 mg/L, 200 mg/L, etc.) formulated under the brand BASTA®. Treatment of plants with glufosinate at 25 mg/L yielded a 100% survival rate, 50 mg/L yielded a 50% survival rate, 150 mg/L yielded a 100% survival rate, and 200 mg/L yielded a 50% survival rate. As can be seen in FIGS. 9-10, the exemplary plant of the invention appears to experience little to no injury as a result of exposure to glufosinate.

FIG. 9: Exemplary herbicide-resistant rubber-producing dandelion plant cultivar HBR-TKS-BAR 1 for use in plant breeding according to the present invention.

FIG. 10: Exemplary herbicide-resistant rubber-producing dandelion plant cultivar HBR-TKS-BAR 2 according to the present invention.

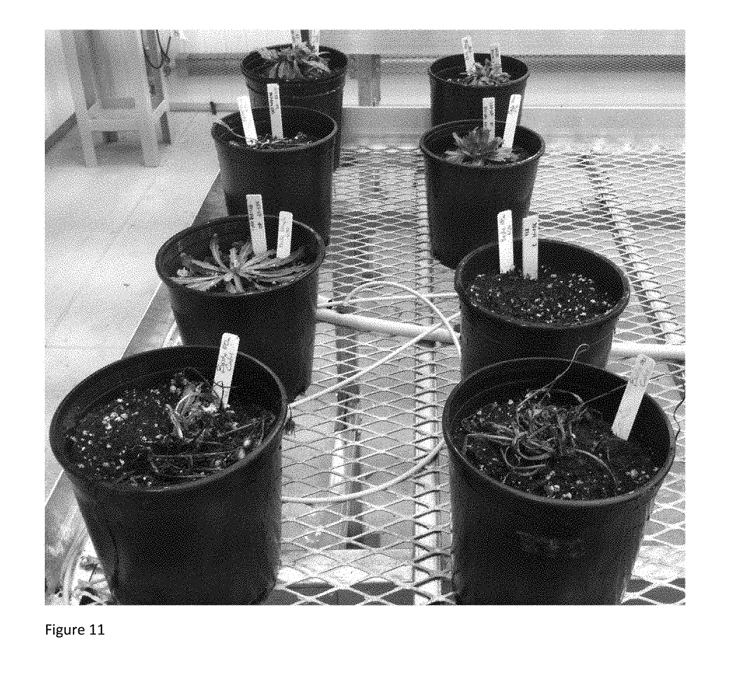

FIG. 11: Comparison of wild-type dandelions and exemplary herbicide-resistant rubber-producing dandelions after exposure to herbicide.

As can be seen in FIG. 11, wild-type plants (front left and front right) display significant injury and/or death when exposed to glufosinate. In contrast, the exemplary dandelions of the present invention display varying degrees herbicide tolerance.

### Example 3—Introducing ALS Herbicide Resistance to Taraxacum kok-Saghyz Using CRISPR

The acetolactate synthase (ALS) inhibiting herbicides, also called acetohydroxyacid synthase (AHAS), have a broad spectrum of selectivity and are readily absorbed by both roots and foliage. ALS herbicides can be translocated in both the xylem and phloem to the site of action at the growing points. ALS has diverse herbicides belonging to different chemistries including: sulfonylureas, imidazolinones, triazolopyrimidines, sulfonyl-aminocarbonyl triazolinones, and pyrimidinyl thiobenzoates. These herbicides inhibit ALS, a key enzyme in the pathway of biosynthesis of the branched-chain amino acids leucine, isoleucine, and valine. Plant death occurs as the ALS inhibiting herbicides starve the plant of branched-chain amino acids and eventually DNA synthesis.

One or multiple amino acid mutations in ALS can lead to resistance to these herbicides by decreasing the affinity of the herbicide to ALS. Applicants target ALS using CRISPR at one or more positions including, for example, Alanine 122, Proline 197, Alanine 205, Tryptophan 574, Serine 653, Aspartic acid 376, Arginine 377, Glycine 654, and combinations thereof.

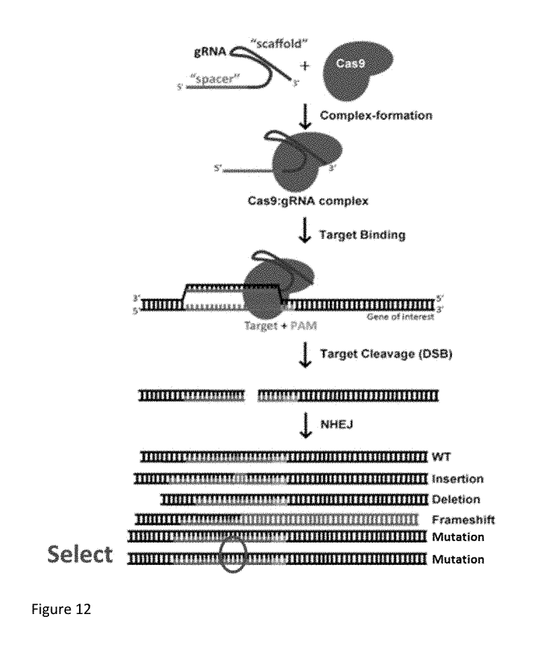

FIG. 12: Plants repair the double strand break by Non-Homologous End Joining (NHEJ) pathway. Nucleotide non-anonymous mutations contributing herbicide resistance could be created and selected.

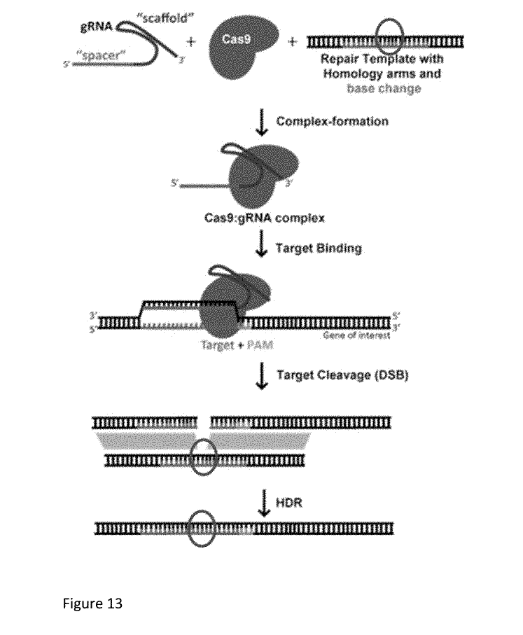

FIG. 13: Plants repair the double strand break by Homology Directed Repair (HDR) pathway. A DNA repair template containing herbicide resistance mutations can be introduced.

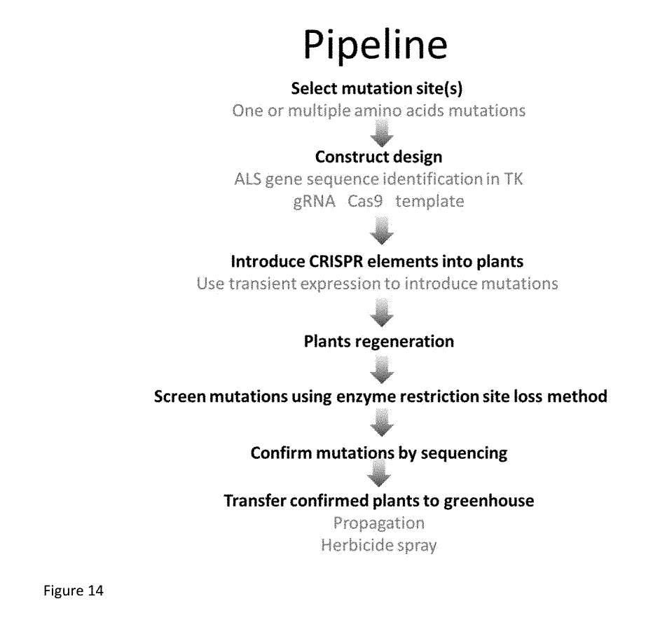

FIG. 14: Schematic employed to generate dandelions with resistance to ALS inhibitors.

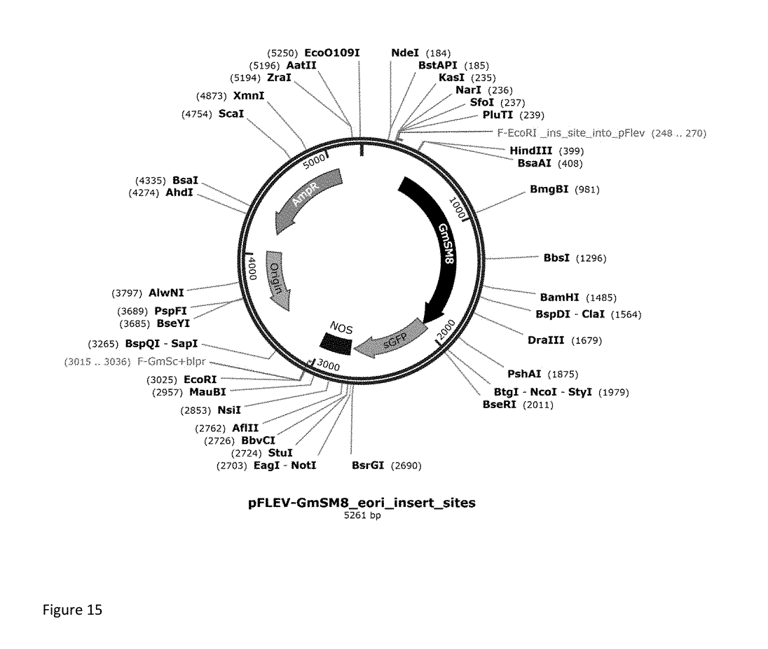

FIG. 15: Plasmid map used to generate the TKS plants of the invention.

TK ALS CRISPR Targets
W574L → W560L SU, IM

| | |
|---|---|
| TCTAGGTATGGTCGTTCAATGGG | (SEQ ID NO: 11) |
| F - GTCGTTCAATgtTttagagctagaaatagc | (SEQ ID NO: 12) |
| cgttcaatGTTTTAGAGCTAGAAATAGCAAG | (SEQ ID NO: 13) |
| R - CATACCTAGAaatcgctatgtcgactctatc | (SEQ ID NO: 14) |
| accatacctagaAATCGCTATGTCGACTCTATC | (SEQ ID NO: 15) |

S653I → A639I/N SU, IM

| | |
|---|---|
| GTGTTGCCTATGATCCCCGCCGG | (SEQ ID NO: 16) |
| F - GATCCCCGCgttttagagctagaaatagc | (SEQ ID NO: 17) |
| gtcgttcaatGTTTTAGAGCTAGAAATAGCAAG - 58° C. | (SEQ ID NO: 18) |
| R - ATAGGCAACACaatcgctatgtcgactctatc | (SEQ ID NO: 19) |
| catacctagaAATCGCTATGTCGACTCTATC- 60° C. | (SEQ ID NO: 20) |
| CCATGAACCCACCGCCGGCGGGG | (SEQ ID NO: 21) |
| F - CGCCGGCGgttttagagctagaaatagc | (SEQ ID NO: 22) |
| accgccggcgGTTTTAGAGCTAGAAATAGCAAG - 58 | (SEQ ID NO: 23) |
| R - GTGGGTTCATGGaatcgctatgtcgactctatc | (SEQ ID NO: 24) |
| gggttcatggAATCGCTATGTCGACTCTATC- 60 | (SEQ ID NO: 25) |
| TTGCCTATGATCCCCGCCGGCGG | (SEQ ID NO: 26) |
| F - CCCGCCGGgttttagagctagaaatagc | (SEQ ID NO: 27) |
| tccccgccggGTTTTAGAGCTAGAAATAGCAAG | (SEQ ID NO: 28) |
| R - TTGCCTATGATCaatcgctatgtcgactctatc | (SEQ ID NO: 29) |
| tcataggcaaAATCGCTATGTCGACTCTATC | (SEQ ID NO: 30) |
| TCCATGAACCCACCGCCGGCGGG | (SEQ ID NO: 31) |
| F - CCGCCGGCgttttagagctagaaatagc | (SEQ ID NO: 32) |
| caccgccggcGTTTTAGAGCTAGAAATAGCAAG | (SEQ ID NO: 33) |
| R - TGGGTTCATGGAaatcgctatgtcgactctatc | (SEQ ID NO: 34) |
| ggttcatggaAATCGCTATGTCGACTCTATC | (SEQ ID NO: 35) |

P197S/H → P183S/H BM (PS)

| | |
|---|---|
| CCATCACCGGCCAAGTTCCCCGG | (SEQ ID NO: 36) |
| F - CCAAGTTCCCgttttagagctagaaatagc | (SEQ ID NO: 37) |
| R - CCGGTGATGGaatcgctatgtcgactctatc | (SEQ ID NO: 38) |
| CGATCATTCTCCGGGGAACTTGG | (SEQ ID NO: 39) |
| F - CGGGGAACTgttttagagctagaaatagc | (SEQ ID NO: 40) |
| R - GAGAATGATCGaatcgctatgtcgactctatc | (SEQ ID NO: 41) |

A122V → A107V IP

| | |
|---|---|
| CTACCCTGGCGGCGCATCCATGG | (SEQ ID NO: 42) |
| F - GCGCATCCAgttttagagctagaaatagc | (SEQ ID NO: 43) |
| R - GCCAGGGTAGaatcgctatgtcgactctatc | (SEQ ID NO: 44) |
| ATCTCCATGGATGCGCCGCCAGG | (SEQ ID NO: 45) |
| F - GCGCCGCCgttttagagctagaaatagc | (SEQ ID NO: 46) |
| R - ATCCATGGAGATaatcgctatgtcgactctatc | (SEQ ID NO: 47) |

G654 → G640b

| | |
|---|---|
| CCTATGATCCCCGCCGGCGGTGG | (SEQ ID NO: 48) |
| F - GCCGGCGGgttttagagctagaaatagc | (SEQ ID NO: 49) |
| R - GGGGATCATAGGaatcgctatgtcgactctatc | (SEQ ID NO: 50) |
| TTGCCTATGATCCCCGCCGGCGG | (SEQ ID NO: 51) |
| F - CCCCGCCGGgttttagagctagaaatagc | (SEQ ID NO: 52) |
| R - ATCATAGGCAAaatcgctatgtcgactctatc | (SEQ ID NO: 53) |

Plant varieties developed as breeding stock with these events were termed HBR-TKS-A, HBR-TKS-B, HBR-TKS-C, HBR-TKS-D, and HBR-TKS-E.
UPPERCASE = Insert
lowercase = Overlap
Indented = NEBase Changer
Underline = Mutation target

### Example 4

CLONING VECTOR COMPLETE SEQUENCE pYZ_GB, complete sequence
(SEQ ID NO: 54)
TCGCGCGTTTCGGTGATGACGGTGAAAACCTCTGACACATGCAGCTCCC
GGAGACGGTCACAGCTTGTCTGTAAGCGGATGCCGGGAGCAGACAAGCCCGTC
AGGGCGCGTCAGCGGGTGTTGGCGGGTGTCGGGGCTGGCTTAACTATGCGGCA
TCAGAGCAGATTGTACTGAGAGTGCACCATATGCGGTGTGAAATACCGCACAG
ATGCGTAAGGAGAAAATACCGCATCAGGCGCCATTCGCCATTCAGGCTGCGCA
ACTGTTGGGAAGGGCGATCGGTGCGGGCCTCTTCGCTATTACGCCAGTTGCCA
TCATTGAGTTTGGAACCCTGAACAGACTGCCGGTGATAAGCCGGAGGAAGGTG
AGGATGACGTCAAGTCATCATGCCCCTTATGCCCTGGGCGACACACGTGCTAC
AATGGCCGGGACAAAGGGTCGCGATCCCGCGAGGGTGAGCTAACTCCAAAAA
CCCGTCCTCAGTTCGGATTGCAGGCTGCAACTCGCCTGCATGAAGCCGGAATC
GCTAGTAATCGCCGGTCAGCCATACGGCGGTGAATCCGTTCCCGGGCCTTGTA
CACACCGCCCGTCACACTATGGGAGCTGGCCATGCCCGAAGTCGTTACCTTAA
CCGCAAGGAGGGGGATGCCGAAGGCAGGGCTAGTGACTGGAGTGAAGTCGTA
ACAAGGTAGCCGTACTGGAAGGTGCGGCTGGATCACCTCCTTTTCAGGGAGAG
CTAATGCTTGTTGGGTATTTTGGTTTGACACTGCTTCACACCCAAAAAGAAGGG
AGCTACGTCTGAGTTAAACTTGGAGATGGAAGTCTTCTTTCGTTTCTCGACAGT
GAAGTAAGACCAAGCTCATGAGCTTATTATCTCAGGTCGGAACAAGTTGATAG
GATCCCCCTTTTTACGTCCCCATGCCCCCTGTGTGGCGACATGGGGGCGAAAA
AAGGAAAGAGAGGGATGGGGTTTCTCTCGCTTTTGGCATAGTGGGCCCCCAGT
GGGGGGCTCGCACGACGGGCTATTAGCTCAGTGGGTAGAGCGCGCCCCTGATA
ATTGCGTCGTTGTGCCTGGGCTGTGAGGGCTCTCAGCCACATGGATAGTTCAAT
GTGCTCATCGGCGCCTGACCCTGAGATGTGGATCATCCAAGGCACATTAGCAT
GGCGTACTCCTCCTGTTCGAACCGGGGTTTGAAACCAAACTTCTCCTCAGGAG
GATAGATGGGGCGATTCAGGTGAGATCCAATGTAGATCCAACTTTCGATTCAC
TCGTGGGATCCGGGCGGTCCGGGGGGGACCACCATGGCTCCTCTCTTCTCGAG
AATCCATACATCCCTTATCAGTGTATGGACAGCTATCTCTCGAGCACAGGTTTA
GGTTCGGCCTCAATGGGAAAATAAAATGGAGCACCTAACAACGCATCTTCACA
GACCAAGAACTACGAGATCACCCCTTTCATTCTGGGGTGACGGAGGGATCATA
CCATTCGAGCCTTTTTTTTTTCATGCTTTTCCCCGAGGTCTGGAGAAAGCTGAA
ATCAATGGGATGTGTCTATTTATCTATCTCTTGACTCGAAATGGGAGCAGGTTT
GAAAAAGGATCTTAGAGTGTCTAGGGTTGGGCCAGGAGGGTCTCTTAACGCCT
TCTTTTTTCTTCTCATCGGATTCACAAAGACTTGCCATGGTAAGGAAGAAGGGG
AGAACAGGCACACTTGGAGAGCGCAGTACAACGGAGAGTTGTATGCTGCGTTC
GGGAAGGATGAATCGCTCCCGAAAAGGAATCTATTGATTCTCTCCCAATTGGT
TGGACCGTAGGTGCGATGATTTACTTCACGGGCGAGGTCTCTGGTTCAAGTCC
AGGATGGCCCAGGAAGTTATGGGCCGCAATGTGAGTTTTTGTAGTTGGATTTG
CTCCCCCGCCGTCGTTCAATGAGAATGGATAAGAGGCTCGTGGGATTGACGTG
AGGGGGCAGGGATGGCTATATTTCTGGGAGCGAACTCCGGGCGAATGAGACC
ACAACGGTTTCCCACTAGAAATAATTTTGTTTAACTTTAAGAAGGAGATATAC
GGCGCGCCATGGTAGATCTGACTAGTAAAGGAGAAGAACTTTTCACTGGAGTT
GTCCCAATTCTTGTTGAATTAGATGGTGATGTTAATGGGCACAAATTTTCTGTC
AGTGGAGAGGGTGAAGGTGATGCAACATACGGAAAACTTACCCTTAAATTTAT
TTGCACTACTGGAAAACTACCTGTTCCGTGGCCAACACTTGTCACTACTTTCTC
TTATGGTGTTCAATGCTTTTCAAGATACCCAGATCATATGAAGCGGCACGACTT
CTTCAAGAGCGCCATGCCTGAGGGATACGTGCAGGAGAGGACCATCTTCTTCA
AGGACGACGGGAACTACAAGACACGTGCTGAAGTCAAGTTTGAGGGAGACAC
CCTCGTCAACAGGATCGAGCTTAAGGGAATCGATTTCAAGGAGGACGGAAAC
ATCCTCGGCCACAAGTTGGAATACAACTACAACTCCCACAACGTATACATCAT
GGCCGACAAGCAAAAGAACGGCATCAAAGCCAACTTCAAGACCCGCCACAAC
ATCGAAGACGGCGGCGTGCAACTCGCTGATCATTATCAACAAAATACTCCAAT
TGGCGATGGCCCTGTCCTTTTACCAGACAACCATTACCTGTCCACACAATCTGC
CCTTTCGAAAGATCCCAACGAAAAGAGAGACCACATGGTCCTTCTTGAGTTTG
TAACAGCTGCTGGGATTACACATGGCATGGATGAACTATACAAAGCTAGCCAC
CACCACCACCACCACGTGTGAGCGATCGCAGTTGTAGGGAGGGATCCTTAATT
AAATGAGCCCAGAACGACGCCCGGCCGACATCCGCCGTGCCACCGAGGCGGA
CATGCCGGCGGTCTGCACCATCGTCAACCACTACATCGAGACAAGCACGGTCA
ACTTCCGTACCGAGCCGCAGGAACCGCAGGAGTGGACGGACGACCTCGTCCGT
CTGCGGGAGCGCTATCCCTGGCTCGTCGCCGAGGTGGACGGCGAGGTCGCCGG
CATCGCCTACGCGGGCCCCTGGAAGGCACGCAACGCCTACGACTGGACGGCCG
AGTCGACCGTGTACGTCTCCCCCCGCCACCAGCGGACGGGACTGGGCTCCACG
CTCTACACCCACCTGCTGAAGTCCCTGGAGGCACAGGGCTTCAAGAGCGTGGT
CGCTGTCATCGGGCTGCCCAACGACCCGAGCGTGCGCATGCACGAGGCGCTCG
GATATGCCCCCCGCGGCATGCTGCGGGCGGCCGGCTTCAAGCACGGGAACTGG
CATGACGTGGGTTTCTGGCAGCTGGACTTCAGCCTGCCGGTACCGCCCCGTCCG
GTCCTGCCCGTCACCGAGATTTGAAAGCTTGAAATTCAATTAAGGAAATAAAT
TAAGGAAATACAAAAAGGGGGGTAGTCATTTGTATATAACTTTGTATGACTTT
TCTCTTCTATTTTTTTGTATTTCCTCCCTTTCCTTTTCTATTTGTATTTTTTTATCA
TTGCTTCCATTGAATTCCGTGTTCTGTGAATAACTTCGTATAGCATACATTATA
CGAAGTTATGAGAAGTCCGTATTTTTCCAATCAACTTCATTAAAAATTTGAATA
GATCTACATACACCTTGGTTGACACGAGTATATAAGTCATGTTATACTGTTGAA
TAACAAGCCTTCCATTTTCTATTTTGATTTGTAGAAAACTAGTGTGCTTGGGAG
TCCCTGATGATTAAATAAACCAAGATTTTTCTAGACATATGGGTCGACATGGA
ACAGAAGTTGATTTCCGAAGAAGACCCCGAGTAGTCGGGAGGATGGCAGAAG
CGGTGATCGCCGAAGTATCGACTCAACTATCAGAGGTAGTTGGCGTCATCGAG
CGCCATCTCGAACCGACGTTGCTGGCCGTACATTTGTACGGCTCCGCAGTGGAT
GGCGGCCTGAAGCCACACAGTGATATTGATTTGCTGGTTACGGTGACCGTAAG
GCTTGATGAAACAACGCGGCGAGCTTTGATCAACGACCTTTTGGAAACTTCGG
CTTCCCCTGGAGAGAGCGAGATTCTCCGCGCTGTAGAAGTCACCATTGTTGTGC
ACGACGACATCATTCCGTGGCGTTATCCAGCTAAGCGCGAACTGCAATTTGGA
GAATGGCAGCGCAATGACATTCTTGCAGGTATCTTCGAGCCAGCCACGATCGA
CATTGATCTGGCTATCTTGCTGACAAAAGCAAGAGAACATAGCGTTGCCTTGG
TAGGTCCAGCGGCGGAGGAACTCTTTGATCCGGTTCCTGAACAGGATCTATTT
GAGGCGCTAAATGAAACCTTAACGCTATGGAACTCGCCGCCCGACTGGGCTGG
CGATGAGCGAAATGTAGTGCTTACGTTGTCCCGCATTTGGTACAGCGCAGTAA
CCGGCAAAATCGCGCCGAAGGATGTCGCTGCCGACTGGGCAATGGAGCGCCTG
CCGGCCCAGTATCAGCCCGTCATACTTGAAGCTAGACAGGCTTATCTTGGACA
AGAAGAAGATCGCTTGGCCTCGCGCGCAGATCAGTTGGAAGAATTTGTCCACT
ACGTGAAAGGCGAGATCACCAAGGTAGTCGGCAAATAATGACTCGAGGCGGC
CGCCTGCAGGTGCTATTGCTCCTTTCTTTTTTTCTTTTTATTTATTTACTGGTATT
TTACTTACATAGACTTTTTTGTTTACATTATAGAAAAAGAAGGAGAGGTTATTT
TCTTGCATTTATTCATGATTGAGTATTCTATTTTGATTTTGTATTTGTTTGGGCT
GCGGGTCAACTGCCCCTATCGGAAATAGGATTGACTACCGATTCCGAAGGAAC
TGGAGTTACATCTCTTTTCCATTCAAGAGTTCTTATGCGTTTCCACGCCCCTTTG
AGACCCCGAAAAATGGACAAATTCCTTTTCTTAGGAACACATACAAGATTCGT
CACTACAAAAAGGATAATGGTAACCTGCGCCAGGGAAAAGAATGGGCCCGGG
GATATAGCTCAGCTGGTAGAGCGCTGCCCTTGCAAGGCAGATGTCAGCGGTTC
GAGTCCGCTTATCTCCACCACTGCGCCAGGGAAAAGAATAGAAGAAGCGTCTG
ACTCCTTCATGCATGCTCCACTTGGCTCGGGGGGATATAGCTCAGTTGGTAGAG
CTCCGCTCTTGCAATTGGGTCGTTGCGATTACGGGTTGGATGTCTAATTGTCCA
GGCGGTAATGATAGTATCTTGTACCTGAACCGGTGGCTCACTTTTTCTAAGTAA
TGGGGAAGAGGACCGAAACATGCCACTGAAAGACTCTACTGAGACAAAGATG
GGCTGTCAAGAACGTCAAGAACGTAGAGGAGGTAGGATGGGCAGTTGGTCAG
ATCTAGTATGGATCGTACATGGACGGTAGTTGGAGTCGGCGGCTCTCCTAGGG
TTCCCTTATCGGGGATCCCTGGGGAAGAGGATCAAGTTGGCCCTTGCGAACAG
CTTGATGCACTATCTCCCTTCAACCCTTTGAGCGAAATGCGGCAAAAGGAAGG
AAAATCCATGGACCGACCCCATCATCTCCACCCCGTAGGAACTACGAGATTAC
CCCAAGGACGCCTTCGGCATCCAGGGGTCACGGACCGACCATAGAACCCTGTT
CAATAAGTGGAACGCATTAGCTGTCCGCTCTCAGGTTGGGCAGTAAGGGTCGG
AGAAGGGCAATCACTCATTCTTAAAACCAGCGTTCTTAAGGCCAAAGAGTCGG
CGGAAAAGGGGGGAAAGCTCTCCGTTCCTGGTTTCCTGTAGCTGGATCCTCCG
GAACCACAAGAATCCTTAGTTAGAATGGGATTCCAACTCAGCACCTTTTGAGT
GAGATTTTGAGAAGAGTTGCTCTTTGGAGAGCACAGTACGATGAAAGTTGTAA
GCTGTGTTCGGGGGGGAGTTATTGTCTATCGTTGGCCTCTATGGTAGAATCAGT
CGGGGGACCTGAGAGGCGGTGGTTTACCCTGCGGCGGATGTCAGCGGTTCGAG
TCCGCTTATCTCCAACTCGTGAACTTAGCCGATACAAAGCTATATGATAGCACC
CAATTTTTCCGATTCGGCGGTTCGATCTATGATTTATCATTCATGGACGTTGAT
AAGATCCATCCATTTAGCAGCACCTTAGGATGGCATAGCCTTAAAATTAAGGG
CGAGGTTCAAACGAGGAAAGGCTTACGGTGGATACCTAGGCACCCAGAGACG
AGGAAGGGCGTAGTAAGCGACGAAATGCTTCGGGGAGTTGAAAATAAGCATA
GATCCGGAGATTCCCGAATAGGTTAACCTTTCAAACTGCTGCTGAATCCATGG
GCAGGCAAGAGACAACCTGGCGAACTGAAACATCTTAGTAGCCAGAGGAAAA
GAAAGCAAAAGCGATTCCCGTAGTAGCGGCGAGCGAAATGGGAGCAGCCTAA
ACCGTGAAAACGGGGTTGTGGGAGAGCAATACAAGCGTCGTGCTGCTAGGCG
AAGCAGTAGAATGCTGCACCCTAGATGGCGAAAGTCCAGTAGCCGAAAGCAT
CACTAGCTTACGCTCTGACCCGAGTAGCATGGGGCACGTGGAATCCCGTGTGA
ATCAGCAAGGACCACCTTGCTGCATTAATGAATCGGCCAACGCGCGGGGAGAG
GCGGTTTGCGTATTGGGCGCTCTTCCGCTTCCTCGCTCACTGACTCGCTGCGCT
CGGTCGTTCGGCTGCGGCGAGCGGTATCAGCTCACTCAAAGGCGGTAATACGG
TTATCCACAGAATCAGGGGATAACGCAGGAAAGAACATGTGAGCAAAAGGCC
AGCAAAAGGCCAGGAACCGTAAAAAGGCCGCGTTGCTGGCGTTTTTCCATAGG
CTCCGCCCCCCTGACGAGCATCACAAAAATCGACGCTCAAGTCAGAGGTGGCG
AAACCCGACAGGACTATAAAGATACCAGGCGTTTCCCCCTGGAAGCTCCCTCG
TGCGCTCTCCTGTTCCGACCCTGCCGCTTACCGGATACCTGTCCGCCTTTCTCCC
TTCGGGAAGCGTGGCGCTTTCTCAATGCTCACGCTGTAGGTATCTCAGTTCGGT
GTAGGTCGTTCGCTCCAAGCTGGGCTGTGTGCACGAACCCCCCGTTCAGCCCG
ACCGCTGCGCCTTATCCGGTAACTATCGTCTTGAGTCCAACCCGGTAAGACAC
GACTTATCGCCACTGGCAGCAGCCACTGGTAACAGGATTAGCAGAGCGAGGTA
TGTAGGCGGTGCTACAGAGTTCTTGAAGTGGTGGCCTAACTACGGCTACACTA
GAAGAACAGTATTTGGTATCTGCGCTCTGCTGAAGCCAGTTACCTTCGGAAAA
AGAGTTGGTAGCTCTTGATCCGGCAAACAAACCACCGCTGGTAGCGGTGGTTT
TTTTGTTTGCAAGCAGCAGATTACGCGCAGAAAAAAAGGATCTCAAGAAGATC
CTTTGATCTTTTCTACGGGGTCTGACGCTCAGTGGAACGAAAACTCACGTTAAG
GGATTTTGGTCATGAGATTATCAAAAAGGATCTTCACCTAGATCCTTTTAAATT
AAAAATGAAGTTTTAAATCAATCTAAAGTATATATGAGTAAACTTGGTCTGAC
AGTTACCAATGCTTAATCAGTGAGGCACCTATCTCAGCGATCTGTCTATTTCGT
TCATCCATAGTTGCCTGACTCCCCGTCGTGTAGATAACTACGATACGGGAGGG
CTTACCATCTGGCCCCAGTGCTGCAATGATACCGCGAGACCCACGCTCACCGG
CTCCAGATTTATCAGCAATAAACCAGCCAGCCGGAAGGGCCGAGCGCAGAAG
TGGTCCTGCAACTTTATCCGCCTCCATCCAGTCTATTAATTGTTGCCGGGAAGC
TAGAGTAAGTAGTTCGCCAGTTAATAGTTTGCGCAACGTTGTTGCCATTGCTAC
AGGCATCGTGGTGTCACGCTCGTCGTTTGGTATGGCTTCATTCAGCTCCGGTTC
CCAACGATCAAGGCGAGTTACATGATCCCCCATGTTGTGCAAAAAAGCGGTTA
GCTCCTTCGGTCCTCCGATCGTTGTCAGAAGTAAGTTGGCCGCAGTGTTATCAC
TCATGGTTATGGCAGCACTGCATAATTCTCTTACTGTCATGCCATCCGTAAGAT
GCTTTTCTGTGACTGGTGAGTACTCAACCAAGTCATTCTGAGAATAGTGTATGC
GGCGACCGAGTTGCTCTTGCCCGGCGTCAATACGGGATAATACCGCGCCACAT
AGCAGAACTTTAAAAGTGCTCATCATTGGAAAACGTTCTTCGGGGCGAAAACT
CTCAAGGATCTTACCGCTGTTGAGATCCAGTTCGATGTAACCCACTCGTGCACC
CAACTGATCTTCAGCATCTTTTACTTTCACCAGCGTTTCTGGGTGAGCAAAAAC
AGGAAGGCAAAATGCCGCAAAAAAGGGAATAAGGGCGACACGGAAATGTTGA
ATACTCATACTCTTCCTTTTTCAATATTATTGAAGCATTTATCAGGGTTATTGTC
TCATGAGCGGATACATATTTGAATGTATTTAGAAAAATAAACAAATAGGGGTT
CCGCGCACATTTCCCCGAAAAGTGCCACCTGACGTCTAAGAAACCATTATTAT
CATGACATTAACCTATAAAAATAGGCGTATCACGAGGCCCTTTCGTC

### Deposits

Applicant(s) will make a deposit of at least 2500 seeds of HBR-TKS-BAR 1 and HBR-TKS-BAR 2, with the American Type Culture Collection (ATCC), Manassas, Va. 20110 USA, ATCC Deposit No. ______. The seeds deposited with the ATCC on ______ will be taken from the deposit maintained by Ohio State University College of Food, Agricultural, and Environmental Sciences, 2120 Fyffe Road, Columbus, Ohio 43210 since prior to the filing date of this application. Access to this deposit will be available during the pendency of the application to the Commissioner of Patents and Trademarks and persons determined by the Commissioner to be entitled thereto upon request. Upon issue of claims, the Applicant(s) will make available to the public, pursuant to 37 CFR 1.808, a deposit of at least 2500 seeds of cultivar 51-7 IPS 1 with the American type Culture Collection (ATCC), 10801 University Boulevard, Manassas, Va. 20110-2209. This deposit of the celery cultivar 51-7 IPS 1 will be maintained in the ATCC depository, which is a public depository, for a period of 30 years, or 5 years after the most recent request, or for the enforceable life of the patent, whichever is longer, and will be replaced if it becomes nonviable during that period. Additionally, Applicants have or will satisfy all the requirements of 37 C.F.R. §§1.801-1.809, including providing an indication of the viability of the sample. Applicants have no authority to waive any restrictions imposed by law on the transfer of biological material or its transportation in commerce. Applicants do not waive any infringement of their rights granted under this patent or under the Plant Variety Protection Act (7 USC 2321 et seq.).

What is claimed is:

1. A dandelion plant having a chromosome comprising:
a transgene/genomic junction comprising a heterologous transgenic insert comprising a promoter that is operably linked to a gene conferring herbicide resistance, wherein the 5′ terminus of said insert overlaps the 3′ terminus of a native dandelion genomic sequence.

2. The dandelion plant of claim 1, wherein said dandelion plant is a rubber-producing dandelion species (Taraxacum kok-saghyz and Taraxacum brevicorniculatum).

3. The dandelion plant of claim 1, wherein said heterologous transgenic insert encodes a herbicide resistance protein selected from the group consisting of a glyphosphate-, ALS-resistance gene (imidazoline, sulfonylurea), aryloxyalkanoate-, HPPD-, PPO-, glufosinate-resistance genes and combinations thereof.

4. The dandelion plant of claim 1, wherein said heterologous transgenic insert encodes the bar gene.

5. A dandelion cell comprising:
a targeted genomic modification to one or more alleles of an endogenous gene in the plant cell, wherein the genomic modification follows cleavage by a site specific nuclease, and wherein the genomic modification produces a mutation in the endogenous gene such that the endogenous gene produces a product that results in an herbicide-resistant dandelion cell.

6. The dandelion cell of claim 5, wherein the genomic modification comprises integration of one or more exogenous sequences.

7. The dandelion plant of claim 1, wherein said dandelion plant is a rubber-producing dandelion species (Taraxacum kok-saghyz and Taraxacum brevicorniculatum).

8. The dandelion cell of claim 6, wherein the exogenous sequence encodes a protein encoding herbicide tolerance.

9. The dandelion cell of claim 8, wherein the exogenous sequence encodes a herbicide resistance protein selected from the group consisting of a glyphosphate-, ALS-resisitance gene (imidazoline, sulfonylurea), aryloxyalkanoate-, HPPD-, PPO-, glufosinate-resistance genes and combinations thereof.

10. The dandelion cell of claim 5, wherein the endogenous gene is an endogenous acetolactate synthase (ALS) gene.

11. The dandelion cell of claim 8, wherein said genomic modification comprises a single-amino acid changes in the ALS protein corresponding to position 197 in Arabidopsis.

12. A method of integrating one or more exogenous sequences into the genome of a dandelion cell, the method comprising:
a) expressing one or more site specific nucleases in the dandelion cell, wherein the one or more nucleases target and cleave chromosomal DNA of one or more endogenous loci;
b) integrating one or more exogenous sequences into the one or more endogenous loci within the genome of the dandelion cell, wherein the one or more endogenous loci are modified such that the endogenous gene is mutated to express a product that results in a selectable phenotype in the dandelion cell; and
c) selecting dandelion cells that express the selectable phenotype, wherein dandelion cells are selected which incorporate the one or more exogenous sequences.

13. The method of claim 12, wherein the one or more exogenous sequences are selected from the group consisting of a insert polynucleotide, a transgene, or any combination thereof.

14. The method of claim 12, wherein integrating the one or more exogenous sequences occurs by homologous recombination or non-homologous end joining.

15. The method of claim 12, wherein the one or more exogenous sequences are incorporated simultaneously or sequentially into the one or more endogenous loci.

16. The method of claim 12, wherein one or more endogenous loci comprise an acetolactate synthase (ALS) gene.

17. The method of claim 16 wherein said modified ALS gene includes changes at a position chosen form the group consisting of Ala122, Pro197, Ala205, Trp574, Ser653, Asp376, Arg377, Gly654, and combinations thereof.

18. The method of claim 12, wherein the site specific nuclease is selected from the group consisting of a CRISPR-Cas single guide RNA nuclease, a zinc finger nuclease, a TAL effector domain nuclease, and a homing endonuclease.

19. The method of claim 18, wherein the site specific nuclease is a CRISPR-Cas single guide RNA nuclease.

20. A method of plant breeding for herbicide resistance in dandelions comprising:
identifying a dandelion plant with a herbicide resistance nucleic acid;
selecting said resistant dandelion plant for use as a parent dandelion plant;
crossing said parent dandelion plant with itself or a second dandelion plant, so that the herbicide resistance trait is passed to progeny seed; and
harvesting progeny seed from said parent dandelion plant.
